(19) United States
(12) Patent Application Publication
(10) Pub. No.: US 2012/0329067 A1
(43) Pub. Date: Dec. 27, 2012

(54) Methods of Generating Zinc Finger Nucleases Having Altered Activity

(76) Inventors: Carlos F. Barbas III, La Jolla, CA (US); Jing Guo, La Jolla, CA (US)

(21) Appl. No.: 13/574,223

(22) PCT Filed: Jan. 21, 2011
(86) PCT No.: PCT/US11/22154

§ 371 Date: Sep. 12, 2012

(30) Foreign Application Priority Data
Jan. 22, 2010 (US) 61297598
Apr. 28, 2010 (US) 61326035

(51) Int. Cl. C12N 9/22; C40B 30/06; C40B 10/00; C12N 15/01; C12P 19/34; C12N 5/09; C12N 15/87; C12Q 1/68; C12N 5/00

(52) U.S. Cl. 435/6.13; 506/10; 506/1; 435/199; 435/375; 435/91.53; 435/462; 435/441

(57) ABSTRACT

Provided herein are zinc linger nucleases having altered, arid in particular, improved catalytic activity and methods of generating such nucleases. Accordingly, there are provided methods for identifying improved catalytic activity of a ZFN by expressing a mutated zinc finger nuclease in a cell containing a reporter construct with a toxic gene, and a zinc finger nuclease cleavage site that is recognized by the ZFN. Survival of the cell is positively correlated with catalytic activity of the ZFN; thus, libraries of mutated ZFKs may be selected for altered catalytic activity based on relative survival rates, Methods of using identified ZFNs are also provided.

### BACKGROUND OF THE INVENTION

1. Field of the Invention

The invention relates generally to zinc finger nucleases having improved catalytic activity and more specifically to methods of generating such nucleases.

2. Background Information

Zinc finger nucleases are chimeric enzymes made by fusing the nonspecific DNA. cleavage domain of the endonuclease FokI with site-specific DNA binding zinc finger domains; these nucleases are powerful tools for gene editing. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied. This method has been applied in many organisms, including plants, Drosophila, C. elegans, zehralish and mammalian cells. These chimeric enzymes can also be used in basic molecular research as other endonucleases are, providing diverse choices for molecular cloning.

The modular structure of C₂H₂ zinc finger motifs and their modular recognition make them an ideal framework for developing custom ZFPs with novel specifity. Each motif recognizes 3 or 4 base pairs via its α-helix and combinations of several ZFs in tandem allow recognition of a long sequence with high specificity. Several approaches have been used to generate ZFPs with high specificity. In the “modular assembly” approach, which had proven to be very effective for the generation of zinc finger transcription factors, ZF motifs with preselected specificities are simply linked together; this is by far the most rapid approach, but is somewhat limited as there are not ZF motifs that recognize each and every one of the 64 DNA triplets. Alternatively, approaches like oligomerized pool engineering (OPEN) have been shown to be effective but require the construction and interrogation of large libraries and have similar sequence limitations. When ZEPs are coupled with the nonspecific FokI old cleavage domain, their affinity and specificity are major determinants of the activity and toxicity of the resulting ZFNs.

Unlike zinc finger transcription factors, which are usually made with six-finger ZFPs that can be designed to recognize a single site within the human genome, ZFNs are typically composed of lower affinity three- or four-finger ZEPs. This is partially due to the nature of ZFN target sites. ZFN target sites are composed of two ZFP binding sites in a tail-to-tail orientation, separated by 5 to 7 bp. Although theoretically every sequence can be targeted by custom ZFPs, in practice, not all can be targeted efficiently. However, three- or four-finger ZFPs, especially those engineered with a modular assembly approach, do not always have sufficient affinity to promote efficient ZFN activity in vivo. Higher activity cleavage domains are therefore desired to improve ZFN activity in vivo.

### SUMMARY OF THE INVENTION

The present invention is based on the design of a directed evolution method to identify ZFNs having enhanced activity, as well as the discovery that ZFNs can enter cells directly without fusion or conjugation to a protein transduction domain. Accordingly, particular embodiments of the invention are directed to methods of identifying DNA cleavage domains having increased catalytic activity, such as a hyperactive FokI cleavage domain (FCD), that can enhance the performance of ZFNs.

Provided herein is an in vivo cell-survival based evolutionary strategy that was utilized to identify a FCD variant called Sharkey, which is 4-5 fold more active than the wild-type enzyme. When coupled with ZFPs, Sharkey stimulated 3-6 fold more mutagenesis in mammalian cells than did ZFNs constructed with the wild-type FokI domain. This novel FCD variant will be useful in future ZFN optimization and applications. Accordingly, the present invention relates to methods of improving the catalytic activity of zinc finger nucleases and methods of use of such nucleases.

In one embodiment, there are provided methods of identifying a DNA cleavage domain (CD) of a zinc finger nuclease (ZFN) having enhanced catalytic activity as compared to a reference ZFN. The method includes expressing a mutated zinc finger nuclease (ZFN) having a DNA cleavage domain (CD) having one or more mutations, and a DNA binding zinc finger domain (ZFD) in a cell comprising a reporter construct. The reporter construct includes in 5′ to 3′ order a promoter, a toxic gene, and a zinc finger nuclease cleavage site that is recognized by the ZFN, such that the toxic gene is operatively linked to the promoter, and whereby the ZFN cleaves the reporter construct, thereby allowing the reporter construct comprising the toxic gene to be degraded. A survival rate is determined for the cell, wherein survival rate is positively correlated with catalytic activity of the CD of the ZFN, and wherein a survival rate for a cell expressing the mutated ZFN that is higher than a survival rate of a cell expressing a reference ZFN is indicative of the CD of the mutated ZFN having enhanced catalytic activity.

In another embodiment of the invention, there are provided methods of identifying a zinc finger nuclease (ZFN) having enhanced catalytic activity. The method includes subjecting a polynucleotide encoding a DNA cleavage domain (CD) to mutagenesis to produce mutated polynucleotides encoding CDs having one or more mutations; fusing the mutated polynucleotides encoding the CDs having one or more mutations to a polynucleotide encoding a DNA binding zinc finger domain (ZFD), thereby creating a library of polynucleotides encoding mutated ZFNs. The library is expressed in cells comprising a reporter construct, wherein the reporter construct comprises in 5′ to 3′ order a promoter, a toxic gene, and a zinc finger nuclease cleavage site that is recognized by the ZFN, wherein the toxic gene is operatively linked to the promoter, and whereby the ZFN cleaves the reporter construct, thereby allowing the reporter construct comprising the toxic gene to be degraded. Cells are selected that express a mutated ZFN having a survival rate that is higher than a survival rate of a cell expressing a reference ZFN, wherein a higher survival rate is indicative of the mutated ZFN having enhanced catalytic activity.

In still another embodiment, there are provided isolated zinc finger nuclease (ZFN) proteins including a zinc finger DNA cleavage domain (CD) having enhanced catalytic activity obtained by a method provided herein, and a DNA binding zinc finger domain (ZFD). In one aspect, the isolated zinc finger nuclease includes a CD having an amino acid sequence selected from the group consisting of SEQ ID NOs:3-6. In some embodiments, the ZED contains three, or four, or more zinc finger proteins. In one aspect, the ZED contains three zinc finger proteins; in another aspect, the ZED contains four zinc finger proteins. Also provided are polynucleotide molecules encoding such ZFNs.

In a further embodiment, there are provided isolated zinc finger nucleases (ZFN) having altered catalytic activity obtained by a method of the invention. In one aspect, the isolated zinc finger nuclease includes the amino acid sequence of SEQ ID NOs: 1 or 2. Also provided are polynucleotide molecules encoding such ZFNs.

In still another embodiment of the invention, there are provided methods of introducing a break into a nucleic acid molecule at a site of interest. The method includes contacting a nucleic acid molecule with a ZFN as provided herein or identified by a method provided herein, or containing a CD provided herein or identified by a method provided herein. The ZFN contains a DNA binding zinc finger domain (ZFD) that binds a target site in proximity to the site of interest so that upon binding of the ZFN to the target site, the ZFN cleaves the nucleic acid at the site of interest, thereby introducing a break into the nucleic acid molecule.

In still another embodiment, there are provided methods of treating a subject having a cell proliferative disorder. The method includes inactivating or mutating a gene according by administering a ZFN as provided herein or identified by a method provided herein, or containing a CD provided herein or identified by a method provided herein to the subject, wherein over-expression of the gene is associated the cell proliferative disorder, thereby treating the cell proliferative disorder.

In yet another embodiment, there are provided methods of producing a cell in which a gene of interest has been mutated. The method includes mutating the gene of interest in a cell or population of cells by introducing, into the cells, a ZFN as provided herein, wherein the ZFN contains a DNA binding zinc finger domain (ZFD) that binds a target site within the gene of interest, such that the ZFN is expressed in the cell, whereby the ZFN binds to the target site and cleaves the gene of interest; and culturing the cells whereby progeny cells in which the gene of interest is mutated are produced. In particular embodiments, the cell is transfected with a nucleic acid molecule encoding the ZFN.

In still another embodiment, there are provided methods of mutating or knocking out a gene of interest in a cell or population of cells. The method includes mutating the gene of interest in a target cell by contacting the cell with a ZFN protein provided herein or a ZFN containing a CD of native or engineered sequence, wherein the ZFD binds a target site within the cell genome, with the proviso that the ZFN is not fused or conjugated to a protein transduction domain, such that the ZFN binds to the target site and cleaves the gene of interest; and culturing the cell, whereby progeny cells in which the gene of interest is mutated or knocked out are produced. In certain embodiments, mutating the gene of interest results in activation or restoration of expression of the gene of interest.

In a further embodiment, there are provided methods of mutating a gene of interest in a cell or population of cells by mutating the gene of interest in a target cell by contacting the cell with a ZFN protein containing a protein transduction domain. The ZFN is as provided herein or containing a CD of engineered sequence, wherein the ZFD binds a target site within the cell genome, and wherein the ZFN is fused or conjugated to a protein transduction domain, such that the ZFN binds to the target site and cleaves the gene of interest and culturing the cell, whereby progeny cells in which the gene of interest is mutated or knocked out are produced. In one aspect, the method further includes delivering to the cell, either prior to, simultaneously with or following mutating the gene of interest, a corrective nucleic acid or vector containing the nucleic acid, thereby providing a substitute for the knocked out or mutated gene of interest.

### BRIEF DESCRIPTION OF THE DRAWINGS

FIG. 1 shows a schematic of the reporter construct (FIG. 1a), the ZFN construct (FIG. 1b), and the selection strategy for identifying ZFNs having altered catalytic activity.

FIG. 2a shows photographs of the colonies resulting from the transformation of a library of ZFNs into selection strain BW25141 and subjected to multiple rounds of evolution. FIG. 2b shows a plot of the survival rate curves for wt, R3, R6, and R9. FIG. 2c shows a photograph of the extent of linearization of the substrate in cellular extracts, FIG. 2d shows a plot of the survival rate measured for each round of selection at 1 hour.

FIG. 3a shows a photograph of the in vitro cleavage of target DNA. by P3 nuclease with either Sharkey or wtFokI catalytic domain. FIG. 3b shows a plot of cleavage rates determined by measuring the initial velocity of pSub-P3 linearization for Sharkey and wt. FIG. 3c shows a three-dimensional structure of full-length FokI in complex with DNA (PDB ID: 1FOK). FIG. 3d shows a diagram depicting the location of S418P and Q481H proximal to Asp450, Asp467 and Lys469 in FokI. FIG. 3e shows activity analysis of FCD variants containing the selected mutations S418P, K441E, Q481H, N527D, and S418P::K441E with the P3 ZF domain. ZFN activity was measured against MluI (ACGGCT) and N×6 (N=A, T, C, or G) spacer sequences and normalized to wild-type FCD. Error bars indicate standard deviation of three replicates. FIG. 3f shows an activity analysis of FCD variants S418P::K441E (Sharkey) and FCDR18-28 (Sharkey') with the P3 ZF domain. ZFN activity was measured against MluI, N×6, ACGAAT, VF2471 (GAGAGT), and CFTR (TGGTGA) spacer sequences and normalized to wild-type FCD. Error bars indicate standard deviation of three replicates.

FIG. 4a shows a schematic overview of the reporter system used to evaluate the efficiency of mutagenesis in mammalian cells (EGFP sense (SEQ ID NO:8) and antisense (SEQ ID NO:9)). FIG. 4b shows representative flow cytometry data for reporter cells transfected with CMV controlled wtFokI and Sharkey cleavage domains with 3, 4, 5 and 6-finger zinc finger DNA binding domains. FIG. 4c shows a plot of the quantification of EGFP positive reporter cells following transfection with ZFN. FIG. 4d shows a photograph of the results of the MluI restriction digest assay of HEK 293 reporter cells transfected with ZFN. ‘Cut’ indicates the presence of unmodified reporter gene. ‘Uncut’ indicates the presence of ZFN modified reporter gene.

FIG. 5 shows plots depicting the efficiencies for ZFN dimerization variants consisting of wild-type and Sharkey cleavage domains.

FIG. 6 shows the sequences of nuclease constructs (SEQ ID NO′S1 to 6). FIG. 6a shows the complete amino acid sequence of the P3.wt construct used in protein evolution and the E6.wt construct used in the mutagenesis assay. The recognition α-helices is underlined. (b) The amino acid sequences of the FokI cleavage domain, Sharkey, Sharkey D483R and Sharkey DAMQS. Amino acids 384 to 579 of the full-length FokI. was used as the cleavage domain. Differences between wild-type and other variants are underlined. Differences in Sharkey relative to wt are underlined bold. Mutations unique to heterodimers are underlined italics.

FIG. 7 shows a plot of the results of a γ-H2AX based cytotoxicity assay.

FIG. 8 shows the amino acid sequence of the E4.FN construct (SEQ ID NO:7).

FIG. 9 shows a schematic overview of the reporter system used to evaluate the efficiency of mutagenesis in mammalian cells in Example 2 (SEQ ID NO′S 8 and 9).

FIG. 10 shows plots of the % EGFP positive cells by FACS analysis.

FIG. 11 shows a photograph of results of an Mini digestion assay.

FIG. 12 shows a schematic of the reporter construct, the ZFN construct, and the selection strategy for identifying ZFNs having altered activity using a negative selection strategy.

FIG. 13a shows an electrostatic potential map of the ZFN surface. FIG. 13b shows an SDS-PADE analysis of purified rZFN consisting of the native FokI (wt) and the Sharkey (Sh) cleavage domain. FIGS. 13c and 13d show flow cytometry analysis of HEK 293 cells following transduction with (c) medium and (d)ᵣZFN.

### DETAILED DESCRIPTION OF THE INVENTION

Before the present methods and compositions are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and the include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention is based on the design of a directed evolution method to identify ZFNs having enhanced activity, as well as the discovery that ZFNs can enter cells directly without fusion or conjugation to a protein transduction domain. Accordingly, particular embodiments of the invention are directed to methods of identifying DNA cleavage domains having increased catalytic activity, such as a hyperactive FokI cleavage domain, that can enhance the performance of ZFNs.

Zinc finger nucleases (ZFNs) are enzymes having a DNA cleavage domain and a DNA binding zinc finger domain. ZFNs may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, the zinc finger nucleases (ZFNs) have altered catalytic activity and are obtained by a method of the invention. In certain embodiments, the ZFN is cell permeable, that is, the ZFN is able to cross the cell membrane when contacted with the cell. In some embodiments, such cell permeable ZFNs are not fused or conjugated to a protein transduction domain. In other embodiments, the ZFN is fused or conjugated to a protein transduction domain. Protein transduction domains (PTDs) as used herein generally refer to polypeptides capable of transducing cargo across the plasma membrane, allowing the proteins to accumulate within the cell. Three exemplary PTDs include the Drosophila homeotic transcription protein antennapedia (Antp), the herpes simplex virus structural protein VP22; and the human immunodeficiency virus 1 (HIV-1) transcriptional activator Tat protein. Additional PTDs are known in the art (e.g., Wadia & Dowdy, Curr Opin Biotech 13:52-6, 2002; Snyder & Dowdy, Expert Opin Drug Deliv 2(1):43-51, 2005) may be fused or conjugated to a ZFN by recombinant or chemical conjugation methods known in the art.

In one aspect, the isolated zinc finger nuclease includes the amino acid sequence of SEQ ID NOs:1 or 2. In other aspects, the ZFN contains a cleavage domain selected from the group consisting of SEQ ID NOs:3-6, In particular aspects, the ZFN has increased catalytic activity relative to a reference ZFN. A reference ZFN as used herein is generally a ZFN having known activity. in one aspect, the reference ZFN contains a wild type cleavage domain; in another aspect the reference ZFN is a mutated ZFN, which is further mutated to form successive generations of mutated ZFNs. The reference ZFN may be the immediately-preceding generation of mutated ZFN, when the mutagenesis and selection steps are repeated one or more times.

A “DNA cleavage domain” or “cleavage domain” (CD) includes one or more polypeptide sequences which possesses catalytic activity for DNA cleavage. A cleavage domain can be contained in a single polypeptide chain or cleavage activity can result from the association of two (or more) polypeptides. In general, a CD can be obtained from any endonuclease or exonuclease. Exemplary endonucleases from which a cleavage domain can be derived include, but are not limited to, restriction endonucleases and homing endonucleases. Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Set USA 91:883-887; Kim et at (1994b) J. Biol. Chem. 269:311,978-31,982. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains. In other embodiments, the CD may be a variant of a wild type cleavage domain. Such variant CDs may contain 1, 2, 3, 4, 5, 6, or more mutations. Such variant CDs may be generated by the methods provided herein.

In some embodiments, the CD may be a wild type FokI cleavage domain (FCD) from endonuclease FokI. In one aspect, the FCD contains the sequence set forth in SEQ ID NO:3.:in other embodiments, the CD may be a variant of the FCD. Such variant FCDs may contain 1, 2, 3, 4, 5, 6, or more mutations. In some embodiments, the FCD has one or more mutations selected from the group consisting of S418P, F432I, K441E, Q481H, H523Y, N527D, and K559Q. in some aspects, the CD contains a sequence as set forth in SEQ ID No:4, 5, or 6.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “DNA binding zinc finger domain” (ZFD) or binding domain is a protein; or a domain within a larger protein, that binds DNA in a sequence-specific-manner through one or more zinc fingers, which are regions of amino acid sequence within the binding domain whose structure is stabilized through coordination of a zinc ion. The term zinc finger DNA binding protein is often abbreviated as zinc finger protein or ZFP. Thus, as used herein, “zinc finger protein,” “zinc finger polypeptide,” or “ZFP” refers to a polypeptide having nucleic acid, e.g., DNA, binding domains that are stabilized by zinc. The individual DNA binding domains are typically referred to as “fingers,” such that a zinc finger protein or polypeptide has at least one finger, more typically two fingers, or three fingers, or even four or five fingers, to at least six or more fingers. In one aspect, the ZFP contains 3 zinc fingers; in another aspect; the ZFP contains 4 zinc fingers. Each finger binds from two to four base pairs of DNA, typically three or four base pairs of DNA. A ZFP binds to a nucleic acid sequence called a target nucleic acid sequence. Each finger usually comprises an approximately 30 amino acids, zinc-chelating, DNA-binding subdomain. An exemplary motif of one class, the Cys2-His2 (SEQ. ID NO:10) class (C₂H2 motif), is —CYS—(X)2-4-CYS—(X)12-HIS—(X)3-5-His (SEQ ID NO:11), where X is any amino acid, and a single zinc finger of this class consists of an alpha helix containing the two invariant histidine residues and the two cysteine residues of a single beta turn that binds a zinc cation (see, e.g., Berg et. al., Science, 271:1081-1085 (1996)). A zinc finger protein can have at least two DNA-binding domains, one of which is a zinc finger polypeptide, linked to the other domain via a flexible linker. The two domains can be identical or different. Both domains can be zinc finger proteins, either identical or different zinc finger proteins.

As used herein, “framework (or backbone) derived from a naturally occurring zinc finger protein” means that the protein or peptide sequence within the naturally occurring zinc finger protein that is involved in non-sequence specific binding with a target nucleotide sequence is not substantially changed from its natural sequence. For example, such framework (or backbone) derived from the naturally occurring zinc finger protein maintains at least 50%, and preferably, 60%, 70%, 80%, 90%, 95%, 99% or 100% identity compared to its natural sequence in the non-sequence specific binding region. Alternatively, the nucleic acid encoding such framework (or backbone) derived from the naturally occurring zinc finger protein can be hybridizable with the nucleic acid encoding the naturally occurring zinc finger protein, either entirely or within the non-sequence specific binding region, under low, medium or high stringency condition. Preferably, the nucleic acid encoding such framework (or backbone) derived from the naturally occurring zinc finger protein is hybridizable with the nucleic acid encoding the naturally occurring zinc finger protein, either entirely or within the non-sequence specific binding region, under high stringency condition.

Zinc finger proteins can be designed and predicted according to the procedures in WO 98/54311 can be used in the present methods. WO 98/54311 discloses technology which allows the design of zinc finger protein domains that bind specific nucleotide sequences that are unique to a target gene. It has been calculated that a sequence comprising 18 nucleotides is sufficient to specify an unique location in the genome of higher organisms. Typically, therefore, the zinc finger protein domains are hexadactyl, i.e., contain 6 zinc fingers, each with its specifically designed alpha helix for interaction with a particular triplet. However, in some instances, a shorter or longer nucleotide target sequence may be desirable. Thus, the zinc finger domains in the proteins may contain at least 3 fingers, or from 2-12 fingers, or 3-8 fingers, or 3-4 fingers, or 5-7 fingers, or even 6 fingers. In one aspect, the ZFP contains 3 zinc fingers; in another aspect, the ZFP contains 4 zinc fingers.

When a multi-finger protein binds to a polynucleotide duplex, e.g., DNA, RNA, PNA or any hybrids thereof, its fingers typically line up along the polynucleotide duplex with a periodicity of about one finger per 3 bases of nucleotide sequence. The binding sites of individual zinc fingers (or subsites) typically span three to four bases, and subsites of adjacent fingers usually overlap by one base. Accordingly, a three-finger zinc finger protein XYZ binds to the 10 base pair site abcdefghij (where these letters indicate one of the duplex DNA) with the subsite of finger X being ghij, finger Y being defg and finger Z being abcd. For example, as known in the art, to design a three-finger zinc finger protein to bind to the targeted 10 base site abcdefXXXX (wherein each “X” represents a base that would be specified in a particular application), zinc fingers Y and Z would have the same polypeptide sequence as found in the original zinc finger discussed above (perhaps a wild type zinc fingers which bind defg and abed, respectively). Finger X would have a mutated polypeptide sequence. Preferably, finger X would have mutations at one or more of the base-contacting positions, i.e., finger X would have the same polypeptide sequence as a wild type zinc finger except that at least one of the four amino residues at the primary positions would differ. Similarly, to design a three-finger zinc protein that would bind to a 10 base sequence abcXXXXhij (wherein each “X” is base that would be specified in a particular application), fingers X and Z have the same sequence as the wild type zinc fingers which bind ghij and abed, respectively, white finger Y would have residues at one or more base-coating positions which differ front those in a wild type finger. The present method can employ multi-fingered proteins in which more than one finger differs from a wild type zinc finger. The present method can also employ multi-fingered protein in which the amino acid sequence in all the fingers have been changed, including those designed by combinatorial chemistry or other protein design and binding assays.

It is also possible to design or select a zinc finger protein to bind to a targeted. polynucleotide in which more than four bases have been altered. In this case, more than one finger of the binding protein must be altered. For example, in the 10 base sequence XXXdefgXXX, a three-finger binding protein could be designed in which fingers X and Z differ from the corresponding fingers in a wild type zinc finger, while finger Y will have the same polypeptide sequence as the corresponding finger in the wild type fingers which binds to the subsite defg. Binding proteins having more than three fingers can also be designed for base sequences of longer length. For example, a four finger-protein will optimally bind to a 13 base sequence, while a five-finger protein will optimally bind to a 16 base sequence. A multi-finger protein can also be designed in which some of the fingers are not involved in binding to the selected DNA. Slight variations are also possible in the spacing of the fingers and framework.

Methods for designing and identifying a zinc finger protein with desired nucleic acid binding characteristics also include those described in WO98/53060, which reports a method for preparing a nucleic acid binding protein of the Cys2-His2 (SEQ ID NO:10) zinc finger class capable of binding to a nucleic acid quadruplet in a target nucleic acid sequence.

Zinc finger proteins useful in the present method can include at least one zinc finger polypeptide linked via, a linker, preferably a flexible linker, to at least a second DNA binding domain, which optionally is a second zinc finger polypeptide. The zinc finger protein may contain more than two DNA-binding domains. The zinc finger polypeptides used in the present methods can be engineered to recognize a selected target site in a gene of choice. Typically, a backbone from any suitable C2H2-ZFP, such as SPA, SPIC, or ZIF268, is used as the scaffold for the engineered zinc finger polypeptides (see, e.g., Jacobs, EMBO J. (1992) 11:4507; and Desjarlais & Berg, Proc. Natl. Acad. Sci. USA (1993) 90:2256-2260). A number of methods can then be used to design and select a zinc finger polypeptide with high affinity for its target. A zinc finger polypeptide can be designed or selected to bind to any suitable target site in the target gene, with high affinity.

Any suitable method known in the art can be used to design and construct nucleic acids encoding zinc finger polypeptides, e.g., phage display, random mutagenesis, combinatorial libraries, computer/rational design, affinity selection, PCR, cloning from cDNA or genomic libraries, synthetic construction and the like. (see, e.g., U.S. Pat. No. 5,786,538; Wu et at, Proc. Natl, Acad. Sci. USA (1995) 92:344-348; Jamieson et al., Biochemistl, (1994) 33:5689-5695; Rebar & Pabo, Science (1994) 263:671-673; Choo & Klug, Proc. Natl. Acad. Sci. USA (1994) 91: 11168-11172; Pomerantz et al., Science, 267:93-96 (1995); Pomerantz et al, Proc. Nail. Acad, Sci. USA (1995) 92:9752-9756; Liu et at, Proc. Nati, Acad, Sci. USA (1997) 94:5525-5530; and Desjarlais & Berg, (1994) Proc. Natl. Acad. Sci. USA 91:11-99-11103).

Zinc finger proteins and zinc finger nucleases can be made by any recombinant DNA technology method for gene construction. For example, PCR based construction can be used. Ligation of desired fragments can also be performed, using linkers or appropriately complementary restriction sites. One can also synthesize entire finger domain or parts thereof by any protein synthesis method. Preferred for cost and flexibility is the use of PCR primers that encode a finger sequence or part thereof with known base pair specificity, and that can be reused or recombined to create new combinations of fingers and ZFP sequences.

The amino acid linker should be flexible, a beta turn structure is preferred, to allow each three finger domain to independently bind to its target sequence and avoid steric hindrance of each other's binding. Linkers can be designed and empirically tested.

If a recognition code is incomplete, or if desired, in one embodiment, the ZFP can be designed to bind to non-contiguous target sequences. For example, a target sequence for a six-finger ZFP can be a nine base pair sequence (recognized by three fingers) with intervening bases (that do not contact the zinc finger nucleic acid binding domain) between a second nine base pair sequence (recognized by a second set of three fingers). The number of intervening bases can vary, such that one can compensate for this intervening distance with an appropriately designed amino acid linker between the two three-finger parts of ZFP. A range of intervening nucleic acid bases in a target binding site is preferably 20 or less bases, more preferably 10 or less, and even more preferably 6 or less bases. It is of course recognized that the linker must maintain the reading frame between the linked parts of ZFP protein.

A minimum length of a linker is the length that would allow the two zinc finger domains to be connected without providing steric hindrance to the domains or the linker. A linker that provides more than the minimum length is a “flexible linker.” Determining the length of minimum linkers and flexible linkers can be performed using physical or computer models of DNA.-binding proteins bound to their respective target sites as are known in the art.

The six-finger zinc finger peptides can use a conventional “TGEKP” (SEQ. ID NO:12) linker to connect two three-finger zinc finger peptides or to add additional fingers to a three-finger protein. Other zinc finger peptide linkers, both natural and synthetic, are also suitable.

A useful zinc finger framework is that of ZIF268 (see WO00/23464 and references cited therein.), however, others are suitable. Examples of known zinc finger nucleotide binding polypeptides that can be truncated, expanded, and/or mutagenized in order to change the function of a nucleotide sequence containing a zinc finger nucleotide binding motif includes TFIIIA and zif268. Other zinc finger nucleotide binding proteins are known to those of skill in the art. The murine CYS2-HiS2 (SEQ ID NO:10) zinc finger protein Zif268 is structurally well characterized of the zinc finger proteins (Pavletich and Pabo, Science (1991) 252:809-817; Elrod-Erickson et al., Structure (London) (1996) 4:1171-1180; and Swirnoff et al., Mol. Cell, Biol, (1995) 15:2275-2287). DNA recognition in each of the three zinc finger domains of this protein is mediated by residues in the N-terminus of the alpha-helix contacting primarily three nucleotides on a single strand of the DNA. The operator binding site for this three finger protein is 5′-GCGIGGGCG-′3. Structural studies of Zif268 and other related zinc finger-DNA complexes (Elrod-Erickson et al., Structure (London) (1998) 6:451-464; Kim and Berg, Nature Structural Biology (1996) 3:940-945; Pavletich and Pabo, Science (1993) 261:1701-1707; Houbaviy et al., Proc. Natl. Acad, Sci, USA (1996) 93:13577-13582; Fairail et al., Nature (London) (1993) 366:483-487; Wuttke et al, J. Mol. Biol. (1997) 273:183-206; Nolte et al., Proc. Nati. Acad. Sci. USA (1998) 95:2938-2943; and Narayan et al, J. Biol. Chem. (1997) 272:7801-7809) have shown that residues from primarily three positions on the α-helix, −1, 3, and 6, are involved in specific base contacts. Typically, the residue at position −1 of the α-helix contacts the 3′ base of that finger's subsite while positions 3 and 6 contact the middle base and the 5′ base, respectively.

However, it should be noted that at least in some cases, zinc finger domains appear to specify overlapping 4 bp sites rather than individual 3 bp sites. In Zif268, residues in addition to those found at helix positions −1, 3, and 6 are involved in contacting DNA (Elrod-Erickson et al., Structure (1996) 4:1171-1180). Specifically, an aspartate in helix position 2 of the middle finger plays several roles in recognition and makes a variety of contacts. The carboxylate of the aspartate side chain hydrogen bonds with arginine at position −1, stabilizing its interaction with the 3′-guanine of its target site. This aspartate may also participate in water-mediated contacts with the guanine's complementary cytosine. In addition, this carboxylate is Observed to make a direct contact to the N4 of the cytosine base on the opposite strand of the 5′-guanine base of the finger 1 binding site. It is this interaction which is the chemical basis for target site overlap.

Any suitable method of protein purification known to those of skill in the art can be used to purify the zinc finger nucleases of the invention (see Sambrook et al., Molecular Cloning: A Laboratory Manual (2ed., Cold Spring Harbor Laboratory Press, Plainview, N.Y.) (1989)). In addition, any suitable host can be used, e.g., bacterial cells, insect cells, yeast cells, mammalian cells, and the like.

As used herein, vector or plasmid refers to discrete elements that are used to introduce heterologous DNA into cells for either expression or replication thereof. Selection and use of such vehicles are well known within the skill of the artisan. An expression vector includes vectors capable of expressing DNAs that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. The present methods include reporter constructs or vectors and ZFN expression constructs or vectors. In one aspect these expression constructs are plasmids.

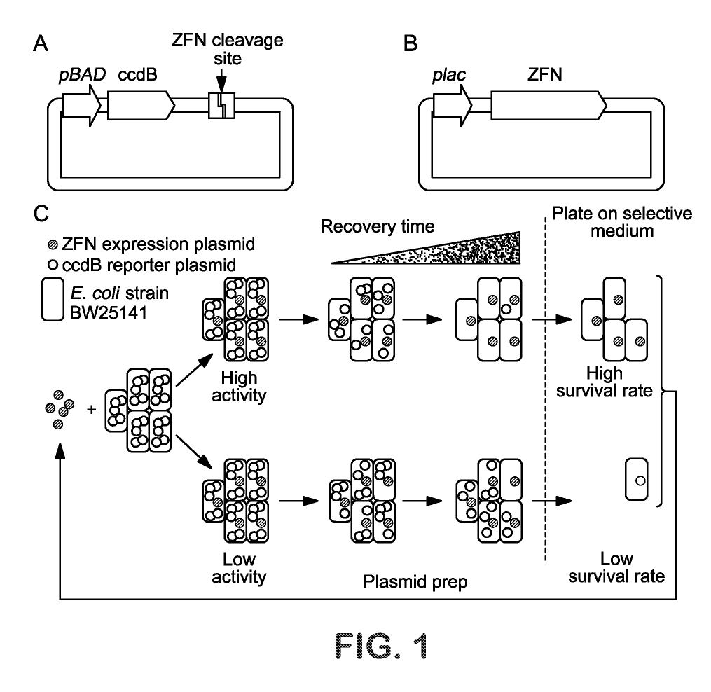

In one embodiment, there are provided methods of identifying a DNA cleavage domain (CD) of a zinc finger nuclease (ZFN) having enhanced catalytic activity as compared to a reference ZFN. The method includes expressing a mutated zinc finger nuclease (ZFN) having a DNA cleavage domain (CD) having one or more mutations, and a DNA binding zinc finger domain (ZFD) in a cell comprising a reporter construct. The reporter construct includes in 5′ to 3′ order a promoter, a toxic gene, and a zinc finger nuclease cleavage site that is recognized by the ZFN, such that the toxic gene is operatively linked to the promoter, and whereby the ZFN cleaves the reporter construct, thereby allowing the reporter construct comprising the toxic gene to be degraded. A survival rate is determined for the cell, wherein survival rate is positively correlated with catalytic activity of the CD of the ZFN, and wherein a survival rate for a cell expressing the mutated ZFN that is higher than a survival rate of a cell expressing a reference ZFN is indicative of the CD of the mutated ZFN having enhanced catalytic activity. In these embodiments, the selection system is a positive selection system. In such a system, mutants that can cleave the reporter construct at the ZFN cleavage site will survive on a corresponding solid selection medium. In one aspect, the reporter construct contains the toxic gene ccdB and a downstream (or 3′) ZFN cleavage site; mutants that cleave at the cleavage sit will survive on solid selection medium (e.g. medium containing 25 ng,/mL zeocin and 10 mM arabinose) (see e.g., FIG. 1).

Recombinant ZFNs (rZFNs) are also used to genetically correct mutations in cells. By way of example, FGFR3 mutations in primary fibroblast cells derived from patients with achondroplasia can be corrected. Current gene delivery methods include plasmid DNA nucleofection, integrase-deficient lentiviral vectors and adenoviral vectors. These strategies, however, require the use of transfection reagents and/or introduction of plasmid DNA that often result in high cellular toxicity. In an effort to establish a safe, non-invasive and efficient method for gene targeting, the present invention provides a protocol for the expression, purification and introduction of cell-permeable rZFNs into human cells.

Positively charged protein transduction domains (PTDs) are frequently used for in vitro and in vivo cellular delivery. Recent reports have demonstrated that engineered superpositively charged proteins can also be used to penetrate human cells and deliver complex molecular payloads. These observations provided the basis for showing that ZFNs can be transduced into mammalian cells for high-efficiency genome modification. Electrostatic potential maps have revealed that the surface of a ZFN is positively charged with isoelectric point (pI)>9.5. Thus, ZFNs may associate with the negatively charged components of the cell membrane in a manner that results in cell penetration. To validate this strategy, the inventors engineered ZFNs to stimulate cleavage and disrupt expression of the HIV-1 co-receptor CCR5. ZFNs designed to target the CCR5 locus were cloned into an expression vector and genetically fused to an N-terminal polyhistidine tag. These enzymes were expressed in E. coli and purified to >90% homogeneity by column chromatography. The conditions have been optimized such that protein re-folding or dialysis is not necessary to restore enzyme activity. To determine whether rZFNs are cell-permeable and can stimulate genomic cleavage in the context of the human cell, we incubated rZFNs with HEK. 293 reporter cells. As described above, these cells have been transformed to contain a nonfunctional EGFP transgene disabled with a ZFN target-site derived from the CCR5 gene. ZFN-mediated mutagenesis of the target allele results in the restoration of EGFP fluorescence. On day 2 after transduction, we analyzed these samples by flow cytometry and found that treatment with rZFNs promoted mutagenesis with efficiencies comparable to that of ZFN expressed by transient transfection. We have since optimized this procedure so that rZFN can stimulate mutagenesis more efficiently than transiently transfected ZFN. addition, we have used rZFNs to disrupt CCR5 expression in CD4+ T cells in culture. Importantly, rZFN entry into cells does not require additional factors such as transfection reagents or viral vectors, thus minimizing toxicity as well as the potential for random integration events. Thus, the data demonstrate that recombinant ZFNs may be used to stimulate mutagenesis in the context of the human genome. In addition, rZFNs may further expand the utility of ZFNs as reagents for routine stem cell modification.

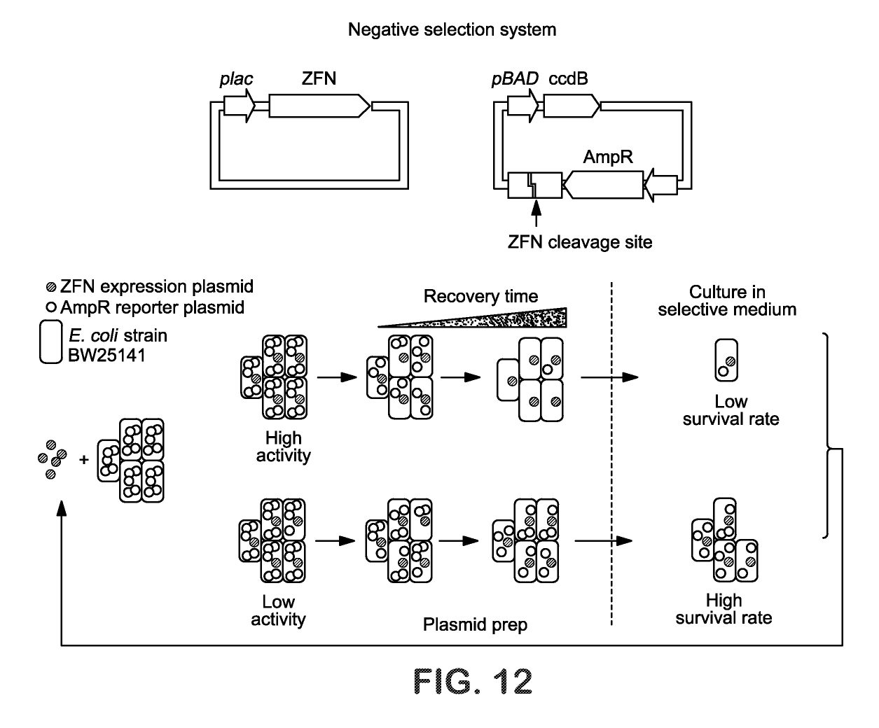

Also contemplated is a negative selection system. In such a system, mutants that can cleave the reporter construct at the ZFN cleavage site will not survive in a corresponding liquid selection medium. In one aspect, the reporter construct contains the ampicillin resistance gene and a downstream (or 3′) ZFN cleavage site; mutants that cleave at the cleavage site will not survive on solid selection medium (e.g., medium containing 25 ng/mL zeocin and 100 μg/mL, carbenicillin) (see e.g., FIG. 12).

The reporter construct or expression construct encoding the ZFN may include a promoter that tightly controls expression of the protein. In one aspect, the BAD promoter is used.

In some embodiments, the determining the survival rate of the cell step is performed by plating on selective medium the cell expressing the mutated ZFN and comparing to the number of colonies produced to the number of colonies produced by plating cells expressing the reference ZFN. Bacterial cells are typically used for these assays. in one aspect E. coli cells are used.

In other embodiments, the method further includes isolating the expression construct encoding the mutated ZFN; mutating the polynucleotide encoding the mutated ZFN to produce a second mutated ZFN, and repeating the expressing and determining steps with the second mutated ZFN to identify altered catalytic activity in the second mutated ZFN, as compared to the mutated ZFN. in one aspect, the catalytic activity is increased as compared to the prior generation of mutated ZFN. This process be repeated one or more times to produce successive generations of mutated ZFNs having further increased activity.

Mutations may be introduced into the polynucleotides encoding the ZFNs by methods known to the skilled artisan. Some of these methods include, random mutagenesis, error-prone PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y.'. (1982)). Random mutagenesis has been the most widely recognized method to date. Typically, this has been carried out either through the use of error-prone PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D F, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation. Random mutagenesis has been the most widely recognized method to date. Typically, this has been carried out either through the use of error-prone PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. In one embodiment, one or more mutations are introduced into a polynucleotide by error-prone amplification (e.g., error-prone PCR) of the polynucleotide.

In another embodiment, mutation of polynucleotides may be achieved by DNA shuffling. DNA shuffling involves the assembly of two or more DNA segments by homologous or site-specific recombination to generate variation in the polynucleotide sequence. In one aspect, DNA shuffling combines the principal of in vitro recombination, along with the method of error-prone PCR. Beginning with a randomly digested pool of small fragments of the polynucleotide, created by Dnase I digestion, random fragments are used in an error-prone PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, because the PCR assembly reaction utilizes error-prone PCR reaction conditions, random mutations are introduced during the DNA synthesis step of the PCR reaction for all of the fragments further diversifying the potential hybridization sites during the annealing step of the reaction.

In still another embodiment of the invention, there are provided methods of introducing a break into a nucleic acid molecule at a site of interest. The method includes contacting a nucleic acid molecule with a ZFN as provided herein or identified by a method provided herein, or containing a CD provided herein or identified by a method provided herein. The ZFN contains a DNA binding zinc finger domain (ZFD) that binds a target site in proximity to the site of interest so that upon binding of the ZFN to the target site, the ZFN cleaves the nucleic acid at the site of interest, thereby introducing a break into the nucleic acid molecule.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor c the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

The site at which the DNA is cleaved generally lies between the binding sites for the two ZFNs. Double-strand breakage of DNA often results from two single-strand breaks, or “nicks,” offset by 1, 2, 3, 4, 5, 6 or more nucleotides, (for example, cleavage of double-stranded DNA by native Fok I results from single-strand breaks offset by 4 nucleotides). Thus, cleavage does not necessarily occur at exactly opposite sites on each DNA strand. In addition, the structure of the ZFNs and the distance between the target sites can influence whether cleavage occurs adjacent a single nucleotide pair, or whether cleavage occurs at several sites. However, for many applications, including targeted recombination and targeted mutagenesis, cleavage within a range of nucleotides is generally sufficient, and cleavage between particular base pairs is not required.

A ZFN can be expressed in a cell following the introduction, into the cell, of polypeptides and/or polynucleotides. Alternatively, a ZFN protein may exert its effect on the chromatin contained within a cell by contacting the cell with the ZFN. In such cases, the ZFN enters the cell and modifies the target gene.

ZFNs for use in the present methods include ZFNs having a DNA cleavage domain (CD) with enhanced catalytic activity obtained by a method provided herein, and a DNA binding zinc finger domain (ZFD). In one aspect, the isolated zinc finger nuclease includes a CD having an amino acid sequence selected from the group consisting of SEQ ID NOs:3-6. In some embodiments, the ZFD contains three, or four, or more zinc finger proteins. In one aspect, the ZFD contains three zinc finger proteins; in another aspect, the ZFD contains four zinc finger proteins. In a further embodiment, the zinc finger nucleases (ZFN) having altered catalytic activity may be obtained by a method of the invention. In one aspect, the isolated zinc finger nuclease includes the amino acid sequence of SEQ ID NOs:1 or 2.

In certain embodiments, targeted cleavage in a genomic region by a ZFN results in alteration of the nucleotide sequence of the region, following repair of the cleavage event by non-homologous end joining (NHEJ). In other embodiments, targeted cleavage in a genomic region by a ZFN can also be part of a procedure in which a genomic sequence (e.g., a region of interest in cellular chromatin) is replaced with a homologous non-identical sequence (i.e., by targeted recombination) via homology-dependent mechanisms (e.g., insertion of a donor sequence comprising an exogenous sequence together with one or more sequences that are either identical, or homologous but non-identical, with a predetermined genomic sequence (i.e., a target site)). Because double-stranded breaks in cellular DNA stimulate cellular repair mechanisms several thousand-fold in the vicinity of the cleavage site, targeted cleavage with ZFNs as described herein allows for the alteration or replacement (via homology-directed repair) of sequences at virtually any site in the genome.

Targeted replacement of a selected genomic sequence requires, in addition to the ZFNs described herein, the introduction of an exogenous (donor) polynucleotide. The donor polynucleotide can be introduced into the cell prior to, concurrently with or subsequent to, expression of the ZFNs. The donor polynucleotide contains sufficient homology to a genomic sequence to support homologous recombination (or homology-directed repair) between it and the genomic sequence to which it bears homology. Approximately 25, 50 100, 200, 500, 750, 1,000, 1,500, 2,000 nucleotides or more of sequence homology (or any integral value between 10 and 2,000 nucleotides, or more) will support homologous recombination. Donor polynucleotides can range in length from 10 to 5,000 nucleotides (or any integral value of nucleotides therebetween) or longer.

It will be readily apparent that the nucleotide sequence of the donor polynucleotide is typically not identical to that of the genomic sequence that it replaces. For example, the sequence of the donor polynucleotide can contain one or more substitutions, insertions, deletions, inversions or rearrangements with respect to the genomic sequence, so long as sufficient homology with chromosomal sequences is present. Such sequence changes can be of any size and can be as small as a single nucleotide pair. Alternatively, a donor polynucleotide can contain a non-homologous sequence (i.e., an exogenous sequence, to be distinguished from an exogenous polynucleotide) flanked by two regions of homology. Additionally, donor polynucleotides can comprise a vector molecule containing sequences that are not homologous to the region of interest in cellular chromatin. Generally, the homologous region(s) of a donor polynucleotide will have at least 50% sequence identity to a genomic sequence with which recombination is desired. in certain embodiments, 60%, 70%, 80%, 90%, 95%, 98%, 99%, or 99.9% sequence identity is present. Any value between 1% and 100% sequence identity can be present, depending upon the length of the donor polynucleotide.

A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

In still another embodiment, there are provided methods of treating a subject having a cell proliferative disorder. The method includes inactivating or mutating a gene according by administering a ZFN as provided herein or identified by a method provided herein, or containing a CD provided herein or identified by a method provided herein to the subject, wherein over-expression of the gene is associated the cell proliferative disorder, thereby treating the cell proliferative disorder. In some embodiments, the ZFN is administered as a protein; in other embodiments, the ZFN is administered as an expression construct encoding the ZFN.

In certain embodiments, the cell proliferative disorder is cancer. A cancer can include, but is not limited to, colorectal cancer, esophageal cancer, stomach cancer, leukemia/lymphoma, lung cancer, prostate cancer, uterine cancer, breast cancer, skin cancer, endocrine cancer, urinary cancer, pancreas cancer, other gastrointestinal cancer, ovarian cancer, cervical cancer, head cancer, neck cancer, and adenomas.

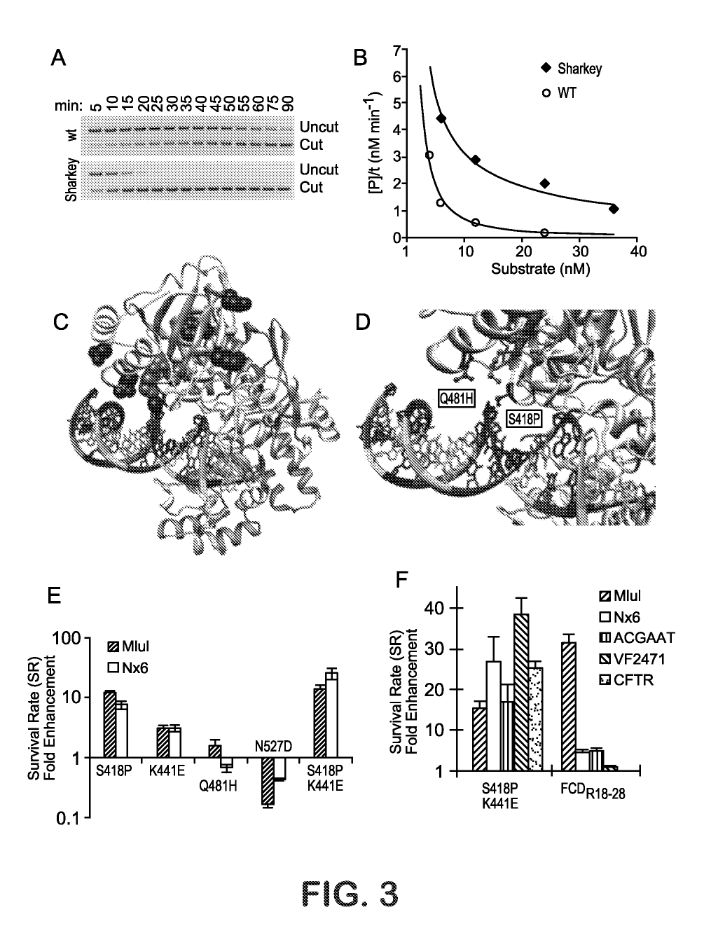

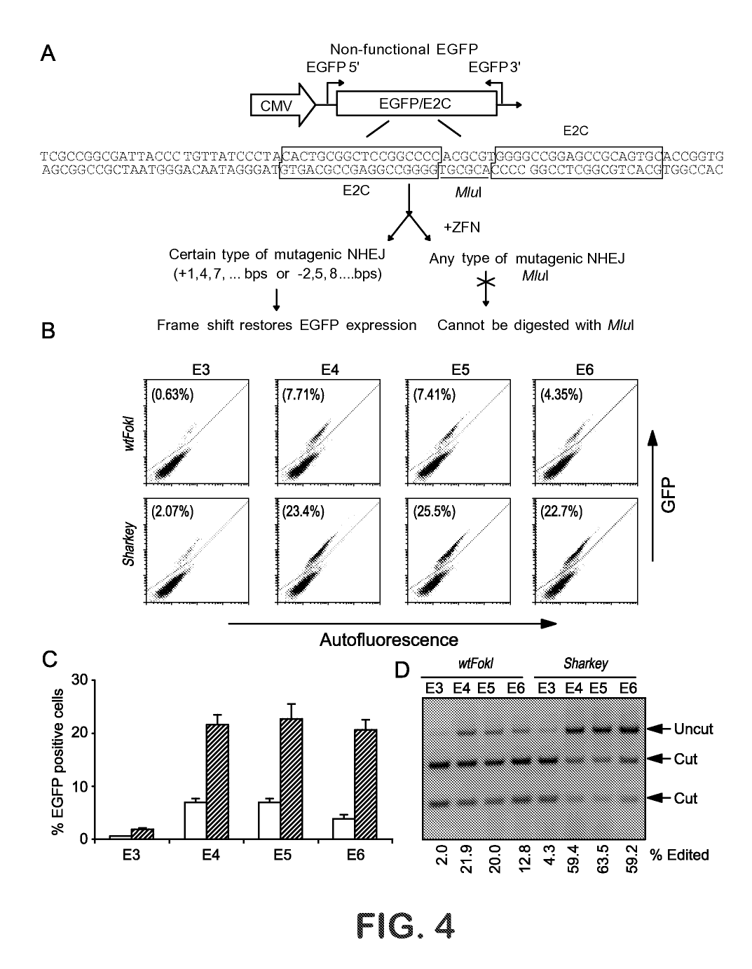

We have improved the efficiency of ZFNs through use of an evolutionary strategy to optimize the catalytic activity of the FokI cleavage domain. Many factors affect the efficiency and precision of a chimeric nuclease, including the specificity and affinity of ZFP (Urnov et al., Nature 435:646-51, 2005; Cornu et al., Mol Ther 16:352-8, 2008; and Smith et al., Nucleic Acids Res 27:778-85, 2007), the length and amino acids sequence of the inter-domain linker (Handel et al., Mol Ther 17:104-11, 2009), the length of spacer DNA in between two ZFP binding sites (Handel et al., Mol Ther 17:104-11, 2009; and Bibikova et al., Mol Cell Biol 21:289-97, 2001) and the interaction between two FokI cleavage domains (Szczepek et al., Nat Biotechnol 25:786-93, 2007; and Miller et al., Nat Biotechnol 25:778-85, 2007). We hypothesized that by increasing the catalytic activity of FCD, we could improve the performance of ZFN. Using the highly sensitive in vivo selection method developed by Zhao et al. (Chen & Zhao, Nucleic Acids 33:e154, 2005), combined with cycling mutagenesis and in vitro DNA shuffling, we were able to identify an FCD mutant, Sharkey, with higher catalytic activity than the wild type domain, as demonstrated by bacterial genetic assays, in vitro DNA cleavage assays, and targeted mammalian genome mutagenesis assays (FIGS. 4). This mutant has seven amino acid substitutions; four of these (S418P, K441E, Q481H, N527D) were present in over 70% of the active clones of the final library. Site-mutagenesis and mammalian NHEJ mediated mutagenesis assays suggested that S418P and Q481H are the critical mutations responsible for the improvement of catalytic activity. These residues are in close proximity to the active center and may contribute in relaxing the active site of the enzyme (FIGS. 3c and 3d). In the course of evolution, we also identified mutations outside the cleavage domain which may contribute to the activity of ZFN by affecting affinity and specificity. Our results suggest that this system may be utilized for optimizing the zinc finger domains of the ZFN as well.

We also optimized the dimer interface via protein evolution. Coupling structure-based redesign with positive antibiotic selection, we were able to select for FCD variants composed of an improved dimeric interface. The dimer interface of wtFokI contains two hydrogen-bonds. The simplest way of converting this symmetric interface into an asymmetric one is to swap one of the hydrogen bonds. However, the resulting hetero-dimer constructs, RR::DD, have much lower activity than wild type enzymes, possibly because the new side-chains are in the proper orientation to form optimal hydrogen bonds. By fixing one partner (RR) and randomizing amino acids on the other partner, we were able to select a new interface, DAMQS, that associated more tightly with the RR interface than did the wild type DD. The resulting hetero-dimer constructs retained high activity. With the increased catalytic activity and compatibility with heterodimer architectures, we anticipate that the Sharkey subdomain will prove generally useful for the growing list of applications of zinc finger nucleases in biology and medicine.

The following examples are intended to illustrate but not limit the invention.

### Example 1

Zinc finger nucleases (ZFNs) are powerful took for gene therapy and genetic engineering. The high specificity and affinity of these chimeric enzymes are based on custom-designed zinc finger proteins (ZFPs). This example illustrates the development of a method to improve the performance of ZFNs, specifically, an in vivo evolution-based approach to improve the efficacy of the FokI cleavage domain (FCD). After cycling mutagenesis and DNA shuffling, a more efficient nuclease variant, termed Sharkey, was generated. An in vitro DNA cleavage assay indicated that the catalytic activity of Sharkey was 4˜5 fold higher than that of the original cleavage domain. A mammalian cell-based assay showed a 3˜6 fold improvement in mutagenesis stimulation for ZFNs containing the Sharkey cleavage domain. Sharkey was compatible with published hetero-dimer architectures.

Plasmid Construction.

Plasmid p11-LacY was used. The P3.FN recognition. sequence was PCR amplified using the primers 5′-CACTCTAGACGCCACTGCACGCGTGCAGTGGCGCTAGGGATAACAGGGTAATATA G-3′ (SEQ ID NO:13) and 5′-CACCACGCATGCCTATATTACCCTGTTATCCCTAG-3′ (SEQ ID NO:14) and was cloned into p11-LacY to generate p11-LacY-sP3/P3, Similarly, the hetero-dimeric E6/P3 recognition sequence was PCR amplified using the primers 5′-CATCTCAGACGCCACTGCACGCGTGGGGCCGGAGCCGCAGTGCTAGGGATAACAG GGTAATATAG-3′ (SEQ ID NO:15) and 5′-CACCACGCATGC CTATATTACCCTGTTATCCCTAG-3′ (SEQ ID NO:16) to generate p11-LacY-sE6/P3. To construct the hetero-dimer reporter plasmid p11-LacY-sE6/P3, an additional expression cassette was PCR amplified from pROLar.A322 and cloned into the NsiI site of p11-LacY-sE6/P3-ΔXbaI. The gene E6FDC9-3 (D483R) was then cloned into the new expression cassette to generate the final reporter plasmid. The expression plasmid pPDAZ was constructed by first removing nucleotides 5-33 from pPRALar.A322 (Clontech Laboratories, Inc.) to generate pPROLar.del.a.ra. This modification abolished arabinose control over protein expression. The zeocin resistance gene was next PCR amplified from pcDNA3.2/zeo(−) and cloned into pPROLar.del.ara with AatII and SacI to form pRDAZ,

Directed Evolution.

Libraries of ZFN mutants were generated by error-prone PCR. Amplification of the FokI cleavage domain was performed over 20 cycles in the presence of 12.5 μM (DTP and 12.5 μM 8-oxo-dGTP. Subsequent overlap PCR was used to fuse cleavage domain (with an average of 4 amino acid mutations) to an error-free copy of P3 ZFP. The resulting ZFN library was cloned into pPDAZ with KpnI and XbaI and electroporated into E. coli. Following transformation, ZFN containing plasmids were isolated from overnight culture and electroporated into the selection strain BW25141. ZFN libraries were routinely composed. of 10⁷˜10⁸ members. Transformed cells were recovered in SOC at 37° C. for 1 hr before plating on solid media containing 25 ng/mL zeocin and 10 mM arabinose. Following 9 rounds of selection, the start codon ATG was replaced with GTG and the recovery time following electroporation was increased to 3 hr. Subsequent rounds of selection saw a decrease in recovery time.

ZFN Purification and in vitro DNA Cleavage Assay.

N-terminal His₆-tagged P3.wt and P3.Sharkey were cloned into the p11-LaCY-wtx1 expression vector, replacing the NheI and XbaI flanked ccdB gene, and transformed into E. coil TOP 10F′. Single colonies were picked and grown in SB media with 90 μM ZnCl₂ and 100 μg/μL carbenicillin at 37° C. with shaking until an OD₆₀₀ of 0.4, at which point each culture was incubated at RT. At an OD₆₀₀ of 0.6, protein expression was induced with 10 mM arabinose. After 5 hrs, cells were harvested via centrifugation and proteins were purified using Ni-NTA agarose resin (Qiagen) and SP Sepharose Fast Flow resin (Amersham Pharmacia Biotech AB). Purified proteins were stored at −80° C. in 50% glycerol until use. The in vitro cleavage assay was performed against linearized substrate plasmid pSub-P3. 12 nM ZFN was added into pre-warmed ZFN Reaction Buffer (20 mM Tris-acetate, 10 mM Magnesium acetate, 50 mM Potassium acetate, 90 μM ZnCl₂, 5 mM 7.9) containing 4, 6, 12, 24, or 36 nM of substrate DNA. Samples were incubated at 37° C. 5-10 μL aliquots were withdrawn at various time intervals. ZFN mediated DNA cleavage was monitored by gel electrophoresis and analyzed with the program ImageJ.

Construction of Mammalian Cell Lines and Measurements of Mutagenesis.

To generate an EGFP reporter gene containing either E6/E6 or E/P4 target sites, a single SgrAI site was inserted between EGFP residues 157 and 158. Synthetic oligonucleotides encoding each target site were subsequently cloned into the SgrAI site. Reporter cell lines composed of a single CMV promoter and a modified EGFP reporter gene were generated in Flp-In-293 cells using the Flp-In system (Invitrogen). Reporter cells were seeded onto polylysine-coated 24-well plate at a density of 1.5×10⁵ per well. After 24 hrs of incubation, these cells were co-transfected with 100 ng ZFN expression plasmid and 500 ng pcDNA3.1/Zeo(−) using LIPOFECTAMINE 2000 transfection reagent (Invitrogen) under conditions specified by the manufacturer. Similarly, for hetero-dimer ZFN assays, cells were co-transfected with 100 ng of each ZFN expression plasmids and 400 ng pcDNA3.1/Zeo(−) carrier DNA. Transfection efficiencies were measured to be between 70˜80%. 3 days post transfection, 30,000 cells were analyzed by flow cytometry (FACScan Dual Laser Flow Cytometer, BD Biosciences) to measure the percentage of EGFP positive cells. Additionally, the rate of mutagenesis was measured by MluI cleavage. Briefly, 3 days post transfection; genomic DNA was harvested and purified with a QIAAMP DNA isolation kit (Qiagen). Modified EGFP gene was amplified in 30 cycles of PCR (Expand High Fidelity, Roche) with 1 ug template DNA, 10% v/v DMSO addition and an annealing temperature of 72 C. The PCR products were digested overnight with Midi and visualized by gel electrophoresis.

γ-H2AX Based Cytotoxicity Assay.

HT1080 cells in 24-well plates were transfected with 100 ng of ZFN or I SceI expression plasmid or the empty expression vector pVAX1 (Invitrogen) plus 500 ng carrier DNA using LIPOFECTAMINE 2000 transfection reagent (Invitrogen). 30 hours post transfection, cells were harvested, stained using the H2A.X phosphorylation assay kit (Millipore) according to the manufacturer's protocol and analyzed by FACS. Alternatively, cells were treated with etoposide at indicated concentrations for 60 min 2h before staining. Etoposide and the reported toxic ZFN construct GZF3.wt28 were used as positive controls, I SceI and pVAX1 were negative controls.

Selection Strategy.

A method for selectively enriching for catalytically improved FokI cleavage domain variants was designed based on a two-plasmid system originally developed by Zhao et al. (Chen & Zhao, Nucleic Acids Res, 33:e154, 2005) for directing activity of the homing endonuclease I SceI to a novel sequence. This system linked DNA cleavage events with cell survival. The reporter plasmid contained the toxic gene ccdB (Bahassi et al., Mol Microbiol 15:1031-7, 1995) under the tightly controlled BAD promoter. Downstream of the ccdB gene was one copy of the desired ZFN cleavage site (FIG. 1a) The low copy ZFN expression plasmid encoded the ZFN gene under the control of a modified lac promoter, which processes an additional lac operator sequence (FIG. 1b) for tighter control on ZFN expression. Cleavage of reporter plasmid by a ZFN mutant linearized the plasmid and caused it to be quickly degraded by RecA. The process includes three steps: 1) expression of endonuclease mutants; 2) cleavage of reporter plasmids by these mutants; 3) degradation of linearized plasmids. Time needed for step three is constant, but that for step two is variable. The DNA cleavage rate of a mutant will influence the rate of step two and, therefore, the time to linearization of the toxic gene. As a consequence, the survival rate (SR) of a mutant, the ratio of the number of colonies on an arabinose selection plate to that on a non-selective plate, was positively correlated with its catalytic activity. Mutants with higher catalytic activity linearize all reporter plasmids in an E. coli cell within a shorter time window and will be enriched during evolution (FIG. 1c).

FIG. 1. Schematic Representation of the Selection Strategy Used for Isolating Novel FCD Variants.

(a,b) A two-plasmid approach utilizing a reporter consisting of a single ZFN cleavage site downstream of ccdB and a ZFN expression plasmid under tight control of a modified lac promoter can be used to selectively enrich for catalytically improved FokI cleavage domains. (c) A library of FCD variants can be transformed into the ccdB harboring BW25141 selection strain and enriched following ZFN mediated reporter plasmid cleavage/degradation. Decreasing the recovery time following transformation facilitates the isolation of ZFN variants with enhanced catalytic properties.

Enhancing the FokI Cleavage Domain.

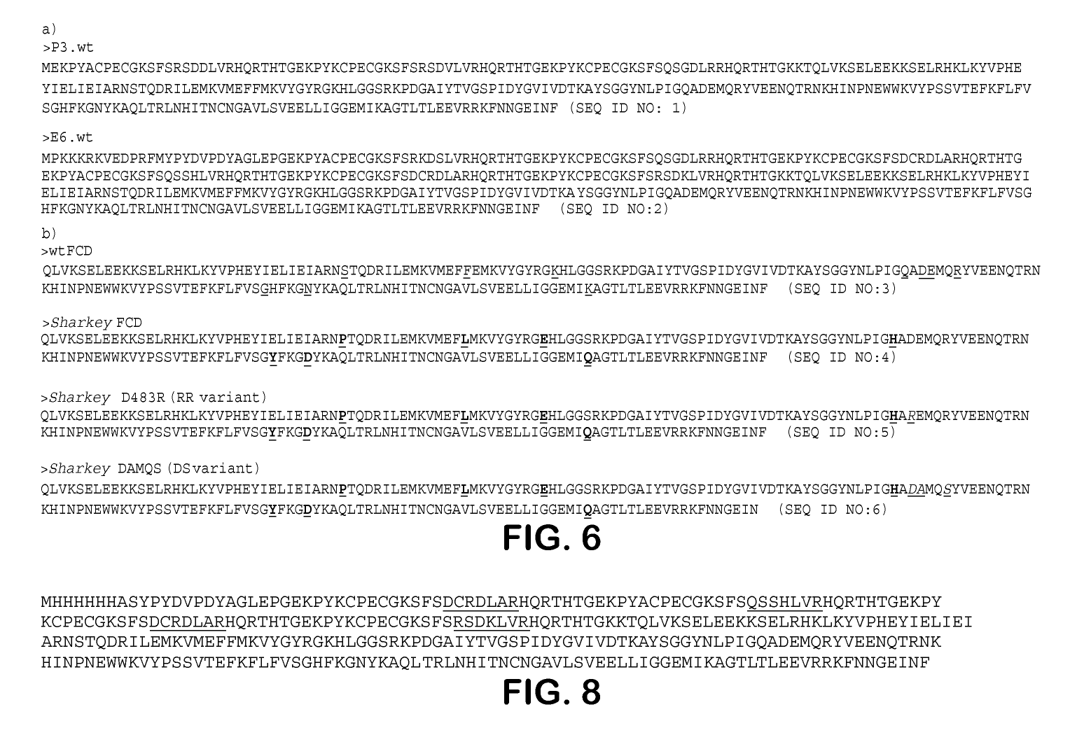

In order to establish proper selective pressure, the SR curve of wild-type (wt) ZFN was first measured. An expression plasmid harboring wt F (AI cleavage domain fused to ZF domain P3 (P3,wt) (FIG. 6) via overlapping PCR was transformed into the strain BW25141 harboring plasmid p11.LacY-sP3/P3. The sequence of the ZFN site in the reporter plasmid p11LacY-sP3/P3 is shown in Table 2. Aliquots of transformants were withdrawn at different time points post transformation and placed on plates grown on solid medium with or without arabinose to calculate the SRs. Without IPTG induction, the resulting SR curve was linear and ˜10% SR was Observed after one hour of recovery. With IPTG induction, however, the reaction was finished within 1 hour and SRs were 80-100%. Therefore, IPTG induction was used for the first round of evolution and no induction for subsequent rounds. The recovery time was 1 hour.

TABLE 1
ZFP binding characteristics and
ZFN target sites

| ZFP | Kd (nM) | ZFP Binding Site |
|---|---|---|
| P3 | — | GCA GTG GCG |
| E3 | 35 | GGG GCC GGA |
| E4 | 10.6 ± 3.3 | GGG GCC GGA GCC |
| | | (SEQ ID NO: 17) |
| E5 | 4.2 ± 1.9 | GGG GCC GGA GCC GCA |
| | | (SEQ ID NO: 18) |
| E6 | 0.85 ± 0.2 | GGG GCC GGA GCC GCA GTG |
| (E2C) | | (SEQ ID NO: 19) |

TABLE 2
Cleavage Site DNA sequence (6 bp spacer)

| | |
|---|---|
| E3/E3 | TCC GGC CCC (ACGCGT) GGG GCC GGA |
| | (SEQ ID NO: 20) |
| P3/P3 | CGC CAC TGC (ACGCGT) GCA GTG GCG |
| | (SEQ ID NO: 21) |
| P3/E6 | CGC CAC TGC (ACGCGT) GGG GCC GGA GCC GCA GTG |
| | (SEQ ID NO: 22) |
| E4/E4 | GGC TCC GGC CCC (ACGCGT) GGG GCC GGA GCC |
| | (SEQ ID NO: 23) |
| E5/E5 | TGC GGC TCC GGC CCC (ACGCGT) GGG GCC GGA GCC GCA |
| | (SEQ ID NO: 24) |
| E6/E6 | CAC TGC GGC TCC GGC CCC (ACGCGT) GGG GCC GGA GCC GCA GTG |
| | (SEQ ID NO: 25) |

FIG. 6. Sequences of Nuclease Constructs.

a) The complete amino acid sequence of the P3.wt construct used in protein evolution and the E6.wt construct used in the mutagenesis assay. The recognition α-helices are underlined, b) The amino acid sequences of the FokI cleavage domain, Sharkey, Sharkey D483R and Sharkey DAMQS (SEQ ID NO:29). Amino acids 384 to 579 of the full-length FokI were used as the cleavage domain. Differences between wild-type and other variants are underlined. Differences in Sharkey relative to wt are in red. Mutations unique to heterodimers are in blue.

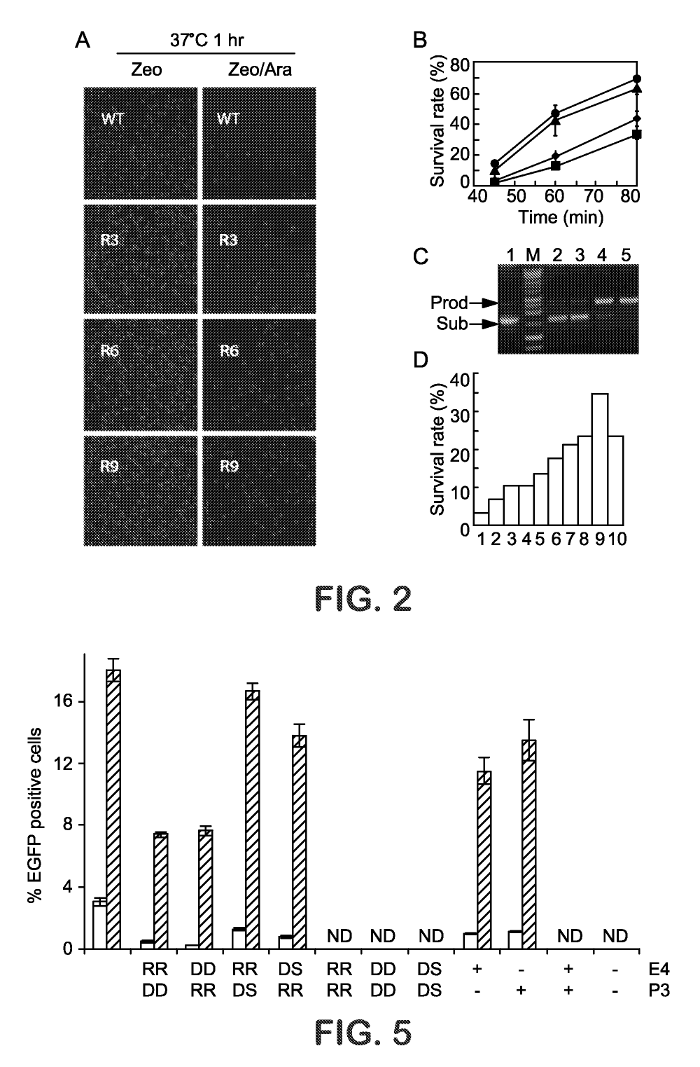

Error-prone PCR was used to introduce diversity into the catalytic domain of FokI prior to fusion to the P3 ZF domain to generate the mutant ZFN library. DNA shuffling (Stemmer, Nature 370:389-91, 1994) was applied to the full ZFN gene to combine beneficial mutations after every three rounds of selection. As the evolution progressed, more clones survived on the selective plates (FIG. 2a). SR curves of rounds 3, 6 and 9 were measured; compared with wt enzyme, rounds 6 and 9 showed much higher SRs at all time points collected (FIG. 2b). A more direct ZFN activity evaluation was carried out in vitro. Supercoiled plasmid pSub-P3, which contained a single copy of ZFN cleavage site, was incubated with cell extracts prepared from rounds 3, 6 and 9 and with the wt ZFN at room temperature. Round 9 cell extracts displayed the highest activity. With this extract, all substrate was linearized within 10 minutes; the reaction of wt ZFN was less than 10% complete at 10 minutes (FIG. 2c).

Because round 9 displayed an SR of over 50% at the 1-hour time point, higher selection stringency was required for further evolution. One way of increasing stringency is to decrease the level of protein expression. The initiation codon of a transcript has a direct impact on its translation efficiency. Changing the start codon from the most frequently used ATG to GTG can reduce protein translation level by five-fold, This strategy was tested on clone L9-3 isolated from the 9^{th} round and it was found that the change in the start codon reduced the SR of this clone from ˜80% to about 8%. A secondary library was then constructed based on the pool of ZFNs from the 9′ round via error prone PCR with the initiation codon switched to GTG, IPTG was added during the 10^{th} round of evolution with this secondary library. Recovery time was initially set at 2.5 hours and shortened by 0.5 hours after every three rounds. Nine more rounds of evolution were carried out with DNA shuffling after every three rounds and the SRs measured at the 1-hour point steadily increased (FIG. 2d).

FIG. 2. Enhancing the FokI Cleavage Domain by Directed Evolution.

A library of ZFNs was transformed into selection strain BW25141 and subjected to multiple rounds of evolution. (a) Survival rate (SR), which correlates directly with catalytic activity, was measured at 1 hr for Rounds 3, 6 and 9. (b) SR was observed to increase with recovery time. SR curves were measured for wt (♦), R3 (▪), R6 (▴) and R9 (), (c) The extent of substrate linearization from Rounds 1-5 was measured from cellular extracts prepared from overnight cultures. ‘Sub’ indicates supercoiled substrate plasmid pSub-P3. ‘Prod’ indicates linearized substrate plasmid pSub-P3. (d) SR was measured for each round of selection at 1 hr. Bar 10 indicates Sharkey cleavage domain.

Sequence Analysis.

During sequence analysis of the last round, it was noticed that vast majority of clones contained mutations in the ZF domain and/or the inter-domain linker in addition to mutations in the FCD. Those mutations were mainly in two areas: the second linker of ZF motif, changing GEKP (SEQ ID NO:26) to GEEP (SEQ ID NO:27), and in the four-amino acid inter-domain linker (GKKT (SEQ ID NO:28) in the wt), mutating one or both lysines to glutamic acid or arginine. These two areas are a distance from the active center and mutations in these regions were likely to have changed the affinity and specificity of the ZFN rather than the catalytic activity. For a more direct comparison of FCD mutants, the FCD of the 18^{th} round was re-amplified, placed it back into the original framework and screened for optimal performance in vivo. One of the most active catalytic domains, termed Sharkey (S418P, F432L, K441E, Q481H, H523Y, N527D, K559Q) was selected for further characterization. With the GTG start codon, this mutant had a 25% SR at 1 hour (FIG. 2d), slightly lower than the overall 33% SR of the 18^{th} but much better than the 8% SR of the best clone from the 9^{th} round and the <1% SR of wt enzyme under the same conditions.

In vitro DNA Cleavage Assay

To determine whether the presence of the Sharkey domain enhanced the DNA cleavage rate, P3 nucleases with either the wt cleavage domain or the Sharkey domain were purified and evaluated in in vitro DNA cleavage assays. Rates of DNA cleavage were determined using a constant concentration of ZFNs (12 nM) and increasing substrate concentrations (from 4 to 36 nM). Linearized plasmid pSub-P3 DNA with a single P3.FN recognition site positioned in the middle of the DNA molecule was used as a substrate. Cleavage of this substrate generates two product DNA molecules of the same size, simplifying the analysis. The progress of each reaction was monitored over time measuring the initial velocity (FIG. 3a). The rate of cleavage for Sharkey was 4-5 fold higher than that of wtFokI, demonstrating that Sharkey had enhance catalytic activity relative to the wt ZFN, Sharkey was also observed to have a faster turnover rate than wtFokI (FIG. 3b).

FIG. 3. Sharkey Has an Enhanced Catalytic Profile as Demonstrated by In Vitro DNA Cleavage.

(a) In vitro cleavage of target DNA by P3 nuclease with either Sharkey or wtFokI catalytic domain. ‘Uncut’ indicates supercoiled substrate plasmid pSub-P3. ‘Cut’ indicates linearized substrate plasmid pSub-P3. Cleavage was monitored incrementally over 90 min. (b) Cleavage rates were determined by measuring the initial velocity of pSub-P3 linearization. (e) Selected Sharkey mutations S418P, F432L, K441E, Q481H, H532Y, N521D and K559Q are depicted as blue spheres on the three-dimensional structure of full-length FokI in complex with DNA (PDB ID: 1FOK). Asp450, Asp467 and Lys469 are depicted as red spheres. (d) S4181) and Q481H are shown to be proximal to Asp450, Asp467 and Lys469, residues important for catalysis in FokI.

Construction of a Mammalian System for Mutagenesis Measurement.

Because one major application of ZFNs is in gene therapy, the catalytic activity of Sharkey was evaluated in a mammalian system. In mammalian cells, the great majority of DSBs are repaired b NHEJ, a somewhat error-prone process, creating small deletions and insertions at. the site of the DSB. It was expected that an increased frequency of DSBs at a given site would lead to increased rate of mutagenesis at this location. A reporter system was constructed to rapidly gauge the potential for ZFNs to create site-specific DSBs. The recognition site for E2C nuclease (Table 1) was inserted into the gene encoding EGEP and subsequently disabled with a frameshift. The resulting nonfunctional transgene was stably integrated at a single location in the genome of HEK 293 cells using the Flp˜IN system (Invitrogen). Certain deletions (e.g., 2, 5, or 8-bp) or insertions 1, 4, or 7-bp) caused by NHEJ mediated mutagenesis restore the frame and hence restore EGFP function (FIG. 4). This assay should reflect a small portion of the total mutation events (about ⅓), but has advantages of no background, robustness and high throughput with little background. Alternatively, the rate of mutagenesis was measured by MluI cleavage assay. If the 6-bp spacer sequence between two ZFP binding sites was that of a MluI restriction site, any mutations in this spacer region would abolish cleavage by and could be evaluated with a limited-cycle PCR/restriction digest assay (FIG. 4).

FIG. 4. Sharkey Increases the Rate of Mutagenesis in a Mammalian Model System.

(a) Schematic overview of the reporter system used to evaluate the efficiency of mutagenesis in mammalian cells. The model system consists of a REX 293 cell line containing a modified and disabled EGFP transgene stably integrated in a single locus, Au MluI restriction site flanked by E2C nuclease recognition sites was inserted between EGFP residues 157 and 158. Select deletions (e.g., 2, 5 or 8-bp) or insertions (e.g., 1, 4, or 7-bp) result in frame restoration and EGFP expression. (b) Representative flow cytometry data for reporter cells transfected with CMV controlled wtFokI and Sharkey cleavage domains with 3, 4, 5 and 6-finger zinc finger DINA binding domains. Mutagenesis is measured by counting the % of EGFP positive cells. (c) Quantification of EGFP positive reporter cells following transfection with ZFN, Error bars denote s.d, (d) MluI restriction digest assay of HEK 293 reporter cells transfected with ZFN. ‘Cut’ indicates the presence of unmodified reporter gene. ‘Uncut’ indicates the presence of ZFN modified reporter gene. The % of modified reporter cells is indicated.

Sharkey Enhances the Efficiency of Mutagenesis in Mammalian Cells.

A series of ZFN containing different numbers of ZF motifs was constructed. ZFP E2C (E6) was a six-finger protein and recognized an 18-bp sequence, whereas ZFPs E5, E4, and E3 were made by deleting one, two or three fingers from the N-termini of the protein, respectively. The affinity of each of the ZFPs for its target was determined by electrophoretic mobility-shift assays (Table 1). ZFNs composed of these ZFPs target the same location in the mammalian genome, simplifying the analysis process and reducing the potential interference caused by background DNA sequence. First, these ZFPs to were fused wtFokI cleavage domain, under the control of a CMV promoter, and compared their abilities to stimulate mutagenesis in mammalian cells by transient expression experiments. On day 3 post transfection, we analyzed these samples with flow cytometry and found that ZFNs with four or five fingers promoted mutagenesis with the highest efficiency (6.87%±0.8% and 7.03%±0.96% EGFP positive, respectively). In the contrast, the activity of E3.wt was barely above background (0.55%±0.10%) and E6.wt (3.96%±0.63%) was only about half as active as E4.wt or E5.wt (FIG. 5a). The low activity of E3.wt was expected, because the affinity of E3 was rather low (35 nM). E6.wt also showed reduced activity even though E6 exhibits appreciable affinity for its target site (0.85 nM). It was possible that too high an affinity may obstruct downstream processes. MluI assays were in agreement with these results (FIG. 5b).

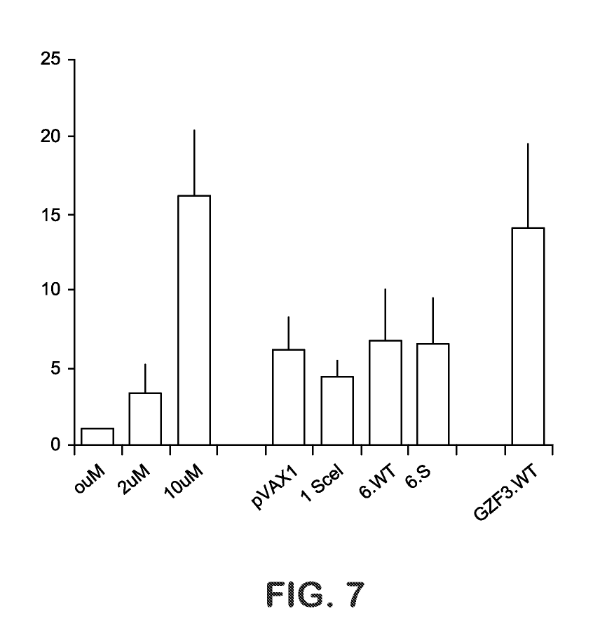

The wt cleavage domain was then substituted in these ZFNs with Sharkey and the same mutagenesis assay was performed. The Sharkey ZFNs increased EGFP expression by 3-6 fold relative to the wt ZFNs (FIGS. 4b and 4c). A 2-3 fold increase was observed by MluI digestion, resulting in up to ˜64% targeted mutatgenesis (FIG. 4d). Sharkey enhanced the performance of E6 nucleases. This may be due to the higher turnover rate of Sharkey. To ensure that the improved activity was not achieved at the cost of increased off-target cleavage, a well-established assay was utilized to measure genome-wide DNA cleavage levels. Phosphorylated histone H2AX (γH2AX) appears rapidly after DNA damage and can be used as a DSB indicator (Rogakou et al, J. Biol Chem 273:5858-68, 1998). Using FITC labeled antibody against γH2AX, the percentage of antibody-stained cells was quantified by flow cytometry. No appreciable difference in the levels of mutation resulting from Sharkey or wt ZFN expression was observed (FIG. 7).

FIG. 7. γ-H2AX Based Cytotoxicity Assay.

HT1080 cells in 24-well plates were transfected with 100 ng of ZFN or I SceI expression plasmid or the empty expression vector pVAX1 (Invitrogen) plus 500 ng carrier DNA using Lipofectamine 2000. 30 hours post transfection, cells were harvested, stained using the H2AX phosphorylation assay kit (Millipore) according to the manufixturer's protocol and analyzed by FACS. Alternatively, cells were treated with etoposide at indicated concentrations for 60 min 2h before staining. Etoposide and the reported toxic ZFN construct GZF3.wt28 are used as positive controls, I SceI and pVAX1 are negative controls. No increase of anti-γ-H2AX staining is observed for E6.Sharkey comparing to E6.wt or other negative controls.

Evolution of Hetero-Dimer Architecture.

The use of ZFNs is often associated with considerable cytotoxicity, a result presumably due to cleavage at off-target sites. One way of reducing off-target cleavage is to convert the homo-dimer interface of FCD into a hetero-dimer interface. Two hetero-dimer interfaces made by structure based redesign have been described: the RR::DD architecture reversed the polarity of the bidendate hydrogen bond between D483 of one ZFN and R487 of its partner (Szczepek et al., Nat Biotechnol 25:786-93, 2007) and the +::−architecture was generated by introducing a positive charge into one ZFN and a negative charge into its partner (Miller et al., Nat Biotechnol 25:778-85, 2007). Although these constructs were as active as wt enzymes for gene targeting, fewer mutations were introduced (Perez et at, Nat Biotechnol 26:808-16, 2008), possibly due to reduced affinity between the cleavage domains. Generation of a better hetero-dimer interface was sought via directed evolution. An extra ZFN expression cassette expressing the E6 nuclease was inserted into the reporter plasmid and replaced the P3/P3 homodimeric cleavage site with an E6/P3 heterodimeric site (Table 2). The best clone from the 9^{th} round, FCD9-3 (S418P, K448E, F1523Y, N527D, R570D), was selected as a starting point, as Sharkey had not yet been evolved when these experiments were begun. To limit the library size, the interface of one ZFN was fixed and the interface of its partner was randomized. ZFN E6.FCD9-3(D483R) was cloned into the reporter plasmid. Positions 483 to 487 of the P3.FCD9-3 in the expression plasmid were NNK randomized to generate the library. Following three rounds of selection, aspartic acid was observed to be the consensus residue at position 483. A smaller library with D483 fixed and positions 484 to 487 NNK randomized was then generated. After five rounds of evolution, the SR was observed to be 27%, a 100-fold improvement relative to P3.FCD9-3(R487D) under the same conditions. Sequence analysis identified the consensus motif DAMQS (SEQ ID NO:29) (referred to as DS) (FIG. 6).

To determine whether Sharkey was compatible with the heterodimer architecture the stimulation of mutagenesis was analyzed by different published heterodimer architectures using an EGFP reporter cell line. This cell line is identical to the previous reporter line described, with the exception of a P3/E4 cleavage site. The pair-wise analysis of these three architectures based on wt enzymes indicated that RR::DD pair retained only one sixth of the wt activity; RR::DS and +::− pair retained about one third of the wt activity. None of the variants showed homodimer activity (FIG. 5). Similar results were observed for Sharkey based heterodimers (FIG. 5), indicating that the Sharkey mutant can be used with any of the described heterodimer architectures.

FIG. 5. Sharkey is Compatible with Alternative ZFN Architectures.

Mutagenesis efficiencies for ZFN dimerization variants consisting of wild-type and Sharkey cleavage domains were determined. The % of EGFP positive reporter cells following transient transfection of ZFN was determined by flow cytometry. ND indicates undetectable. Error bars denote s.d.

Accession Numbers

The nucleic acid sequence of the Sharkey cleavage domain has been deposited in GenBank with accession number HM130522.

### DISCUSSION

We have improved the efficiency of ZFNs through use of an evolutionary strategy geared towards optimizing the catalytic activity of the FokI cleavage domain. Numerous factors affect the proficiency of a chimeric nuclease, including the specificity and affinity of ZFP_{S6, 27}, the length and composition of the inter-domainilinker29, 30, the length of spacer sequence DNA inbetween ZFP binding sites29, 30 and the interaction between two FokI cleavage domains_{31, 32}. We hypothesized that by increasing the catalytic activity of FCD through directed protein evolution; we could improve the performance of ZFNs even if they were constructed using lower affinity ZFPs. Using a sensitive in vivo selection methodology in parallel with cycling mutagenesis, in vitro DNA shuffling, and site-directed mutagenesis we were able to identify a FCD mutant with enhanced catalytic activity relative to the wild-type domain, as demonstrated by bacterial genetic assays, in vitro DNA cleavage assays, and targeted mammalian genome mutagenesis assays (FIG. 24).

Sharkey, the most efficient FokI cleavage domain variant we interrogated, consists of two amino acid mutations, S418P and K441E, which were shown to independently enhance the cleavage capabilities of wild-type FCD. Situated within 8 Å of the catalytic center Lys 469 and within 4 Å of the phosphate backbone of bound substrate DNA (FIG. 6), S418P was observed to increase activity >10-fold relative to wild-type FCD. In comparison, K441E increased catalytic performance >5-fold, relative to wild-type FCD (FIG. 3c). The S418P mutation appears at a turn region in the protein and may contribute to the observed increase in catalytic efficiency by finetuning the structure of the enzyme. The introduction of Pro at the onset of helix 3 may influence its structural rigidity and enable rapid substrate turnover and recognition. In addition to the advantageous S418P and K441E mutations, both Q481H and N527D were observed in ≧70% of sequenced FCD variants. However, while Q481H lies within 7 Å of the catalytic center (FIG. 6), introduction of either itself or N527D to FokI diminishes activity between 2 to 8-fold, relative to the wild-type cleavage domain. Indeed, it appears the accumulation of either of these point mutations over the course of our Sharkey' evolutions resulted in preferential activity, specifically towards the MluI core sequence. The ability to refine the substrate specificity of the FCD by identification of mutations that contribute to substrate discrimination may have great utility for various endogenous gene-targeting applications. Saturation mutagenesis of Gln 481 and Asn 527, two amino acids that may play a role DNA recognition, as well as neighboring amino acids may yield the identification of selective FCD variants capable of discriminating between highly homologous endogenous target sites, thus refining overall specificity in conjunction with the zinc finger domains through cooperative specificity₄₂. The development of ZFNs with preferential FCD activities may further reduce off-target cleavage events and consequently toxicity.

Additionally, over the course of these evolutions, we were able to identify mutations outside the cleavage domain, which may contribute to the activity of ZFNs by affecting the affinity and specificity of custom ZFPs. Our results suggest that our adapted selection system may be utilized for optimizing the zinc finger domains of the ZFN as well. In addition to demonstrating the enhanced cleavage capabilities of Sharkey in a variety of contexts, including a mammalian mutagenesis assay, we showed that Sharkey mutations are compatible with the ZFN architectures developed by Miller et al. and Szczepek et al.

Moreover, towards the goal of improving the efficiency of existing asymmetric ZFN scaffolds, we were successfully able to amend our selection system for the directed evolution of novel heterodimeric architectures. Utilizing saturation mutagenesis to target two critical hydrogen bonds within the dimer interface of Fold, we were able to identify a novel FokI interface, DAMQS, that in conjunction with the D483R ZFN architecture, was able to stimulatemutagenesis more efficiently than the RR::DD ZFN scaffolds developed by Szczepek et al. We suspect the original interface contained sub-optimal hydrogen bonding networks as a result of the in silico based approach used to generate the asymmetric interface. We believe targeting neighboring amino acids by saturation mutagenesis enabled us to experimentally survey favorable regions of sequence space inaccessible through computational approaches and identify increasingly stable ZFN heterodimers with optimal side-chain configurations.

In summary, with increased catalytic capabilities and compatibility with the promising and potentially powerful heterodimeric ZFN scaffolds, we anticipate the Sharkey domain will prove indispensable for a growing list of zinc finger nuclease related applications throughout biology and medicine. (Guo et al., J. Mol. Biol. (2010) 400, 96-107; herein incorporated by reference in its entirety).

### Example 2

The complete amino acid sequence of the E4.FN construct used in this experiment is shown in FIG. 8. The recognition α-helices are underlined. Recombined protein was expressed in E. coli/and purified with a His-tag affinity column and a SP sepharose column. Purified protein was stored at −80° in 50% glycerol until use.

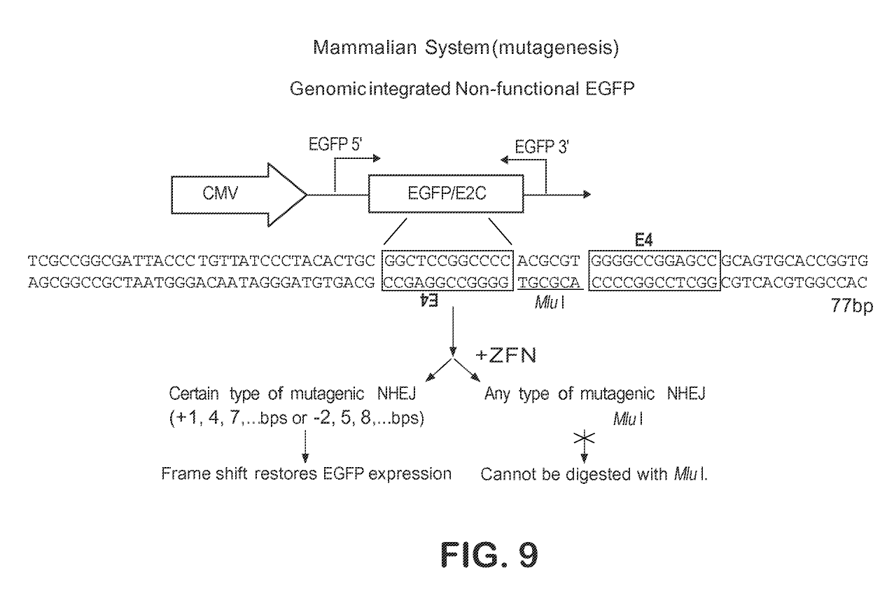

A 77-bp sequence containing the E4.FN recognition site was inserted in between EGFP residues 157 and 158, with the C-terminal part of the coding sequence out of frame (FIG. 9). introducing cell permeable ZFN leads to cleavage at the sequence in between two ZFP binding sites. Certain types of NHEJ-mediated insertions or deletions restore the reading frame and EGFP function. Mutations between the two ZFP binding sites will also destroy the MluI site (FIG. 11).

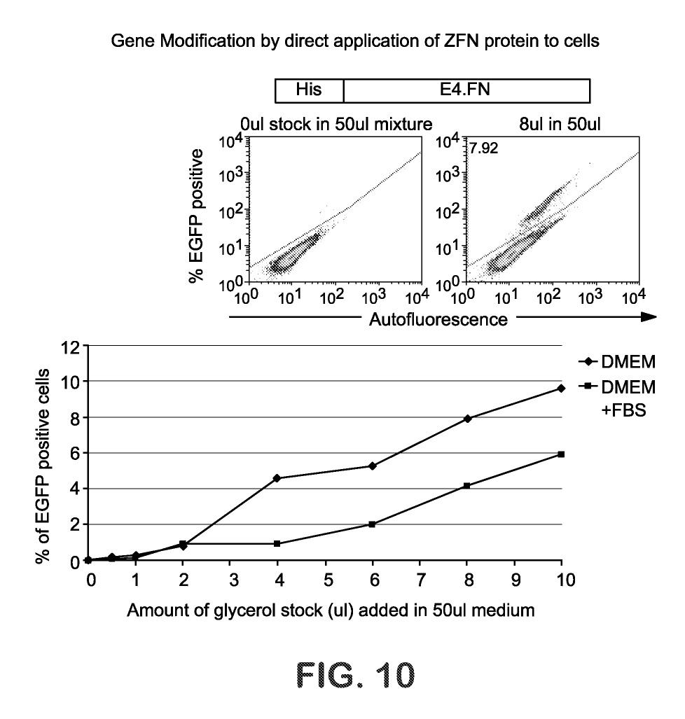

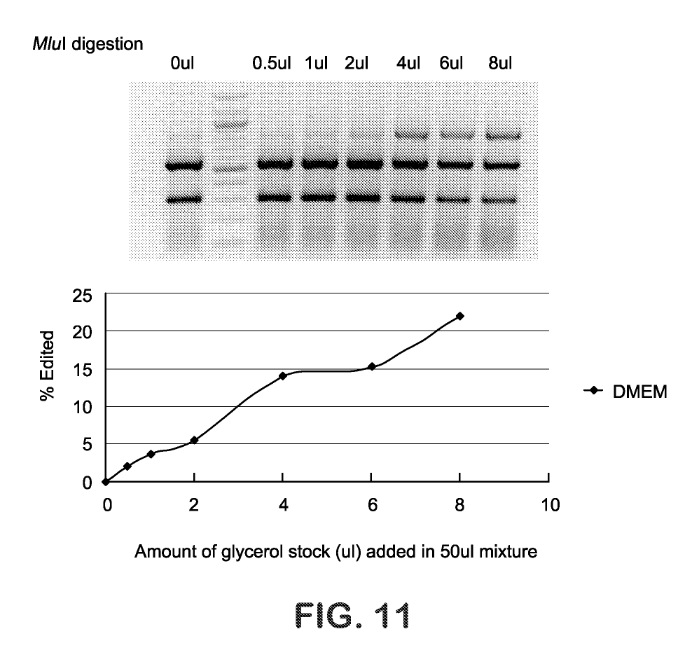

ZFN glycerol stock was diluted with DMEM with or without 10% FBS. Reporter cells were treated with E4.FN in concentrations as indicated for 3 hours 3 days before FACS analysis (FIG. 10). We discovered the unexpected finding that E4.FN lacking fusion or conjugation to a protein transduction domain (PTD) was able to enter cells directly and modify the target gene by cleavage. As shown in FIG. 10, ZFN E4.FN action in cells restores the reading frame of the EGFP gene resulting in enhanced fluorescence in a significant portion of cells exposed to the E4.FN protein. This result is similar to results obtained by fusion of E4.FN to the well-known TAT or polyarginine PTDs (Snyder & Dowdy, Expert Opin Drug Deliv 2(1):43-51, 2005). Other ZFNs also lacking fusion or conjugation to PTDs were also active in entering living cells and mutating targeted nucleic acids. The unexpected finding of the direct activity of ZFNs applied as proteins to enter cells directly upon application without the assistance of a PTD allows for their direct application to cells for targeted nucleic acid modification.

Positively charged protein transduction domains (PTDs) are frequently used for in vitro and in vivo cellular delivery35. Recent reports have demonstrated that engineered superpositively charged proteins can also be used to penetrate human cells and deliver complex molecular payloads36. These observations have inspired our laboratory to examine whether engineered ZFNs can be transduced into mammalian cells for high-efficiency genome modification. Electrostatic potential maps have revealed that the surface of a ZFN is positively charged (FIG. 4A), with an isoelectric point (pI)>9.5. Thus, we hypothesized that ZFNs may associate with the negatively charged components of the cell membrane in a manner that results in cell penetration. To validate this strategy, we engineered ZFNs to stimulate cleavage and disrupt expression of the HIV-1 co-receptor CCR5, ZFNs designed to target the CCR5 locus were cloned into an expression vector and genetically fused to an N-terminal polyhistidine tag. These enzymes were expressed in E. coli and purified to >90% homogeneity by column chromatography (FIG. 4B). Importantly, we have optimized these conditions such that protein re-folding or dialysis is not necessary to restore enzyme activity. To determine whether rZFNs are cell-permeable and can stimulate genomic cleavage in the context of the human cell, we incubated rZFNs with HEK 293 reporter cells. As described above, these cells have been transformed to contain a nonfunctional EGFP transgene disabled with a ZFN target-site derived from the CCR5 gene. ZFN-mediated mutagenesis of the target allele results in the restoration of EGFP fluorescence. On day 2 after transduction, we analyzed these samples by flow cytometry and found that treatment with rZFNs promoted mutagenesis with efficiencies comparable to that of ZFN expressed by transient transfection (FIG. 4C). We have since optimized this procedure so that rZFN can stimulate mutagenesis more efficiently than transiently transfected ZFN. In addition, we have used rZFNs to disrupt CCR5 expression in CD4+ T cells in culture. Importantly, rZFN entry into cells does not require additional actors such as transfection reagents or viral vectors, thus minimizing toxicity as well as the potential for random integration events. We believe these preliminary data demonstrate that recombinant ZFNs may be used to stimulate mutagenesis in the context of the human genome and the FGFR3 locus. In addition, rZFNs may further expand the utility of ZFNs as reagents for routine stem cell modification.

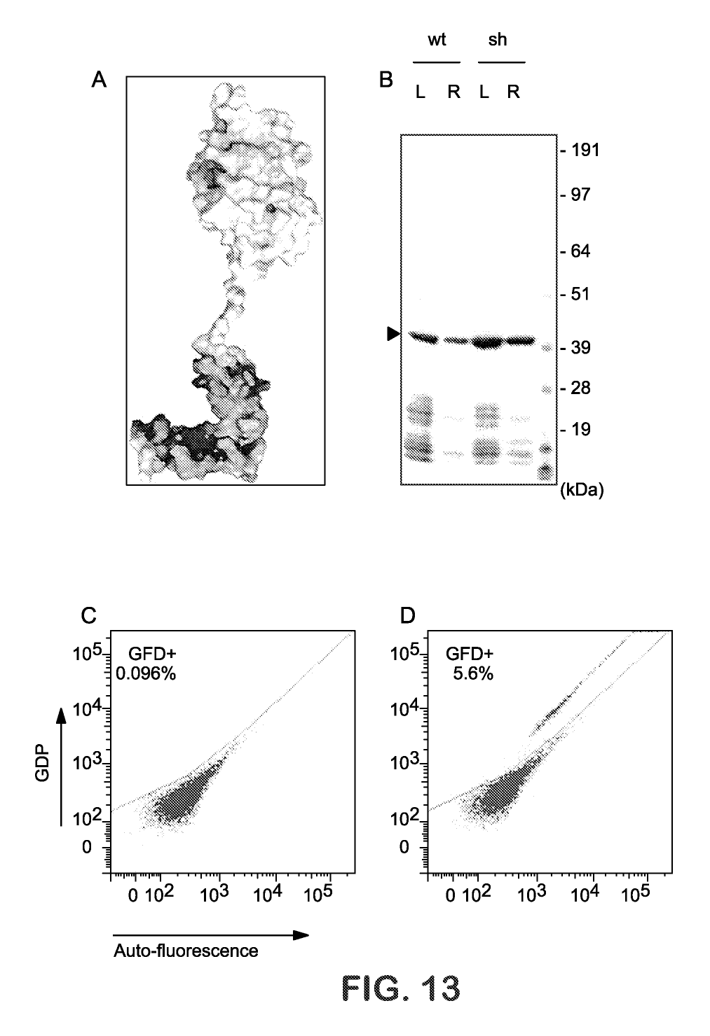

FIG. 13.

Recombinant ZFNs can efficiently stimulate mutagenesis in the human genome, (A) Electrostatic potential map of the ZFN surface. Positively charged residues are depicted blue. Negatively charged residues are depicted red. (B) SDS-PADE analysis of purified rZFN consisting of the native FokI (wt) and the Sharkey (Sh) cleavage domain. (C-D) Flow cytometry analysis of HEK 293 cells following transduction with (C) medium and (D) rZFN.

### Example 3

Traditional methods of gene delivery rely extensively on the use of modified viruses such as adenovirus, lentivirus and retrovirus. Although these viral strategies may be effective at transporting DNA into primary cells or stem cells, these methods are often associated with toxicity and chromosomal mutagenesis. In contrast, non-viral delivery methods are non-invasive and safe but are often hampered by either low efficiency or low activity in vivo. To address these issues, we have developed a strategy that circumvents these limitations and permits the safe and efficient modification of cells by exogenously expressed and purified recombinant ZFNs (rZFNs). An illustrative system uses this strategy to stimulate gene targeting at the FGFR3 locus in fibroblast cells derived from a patient with achondroplasia.

Effective ZFNs are cloned into an expression vector and genetically fused to an N-terminal polyhistidine tag. As described above, these ZFNs will be expressed in E. coli and purified to homogeneity by column chromatography. To ensure that rZFNs can stimulate mutagenesis against the FGFR3 locus, we will analyze enzyme activity in vitro using the EGFP reporter assay described in Aim 1. Fibroblast cells derived from an achondroplasia patient will be obtained from the Coriell Cell Repositories (CCR) at the Coriell. Institute for Medical Research (Catalog ID: GM08858). These cells will be heterozygous for the G380R amino acid mutation in the transmembrane domain of FGFR3. Fibroblast cells will be cultured in Eagle's Minimum Essential Medium (MEM) in the presence of Earle's salts and non-essential amino acids. We will introduce rZFN and donor plasmid into patient cells by incubation at 37° C. in MEM. Recent studies have revealed that when incubated with plasmid DNA, positively charged protein can effectively deliver DNA. by endocytosis. Thus, rZFN and donor plasmid will be pre-mixed and internalized concurrently. After incubation, fibroblast cells will be washed to remove surface-bound protein. The efficiency and specificity of ZFN-induced modification of FGFR3 will be analyzed by a variety of methods including limited-cycle PCR and restriction digestion, Southern blot analysis and genomic PCR of bulk and isolated clonal populations. Moreover, deep-sequencing analysis of genomic PCR products will be used to quantitatively determine if rZFN-driven gene editing results in modification of sequences other than the intended target. Cellular toxicity resulting from non-specific DSBs will be addressed by monitoring the phosphorylation of γH2AX. In the event that donor plasmid is unable to be internalized with rZFN, we will use a biodegradable nanoparticulate polymeric vector to introduce donor plasmid after incubation with rZFN44.

The iPSC-based models of human achondroplasia described herein has utility in high-throughput screening applications aimed at identifying novel compounds that can be used to treat skeletal dysplasia. In particular, these iPSC-based models will permit analysis of the efficacy of thousands of complex small molecule compounds at various development stages during chondrogenesis and bone development. In addition, these methods will allow the generation of iPSC-based models of different types of skeletal dysplasia including hypochondroplasia and thanatophoric dysplasia. These experiments will provide further insight into the mechanisms controlling chondrocyte differentiation, bone development and disease pathophysiology.

Although the invention has been described with reference to the above example, it will be understood that modifications and variations are encompassed within the spirit and scope of the invention. Accordingly, the invention is limited only by the following claims.

What is claimed is:

1. A method of identifying a DNA cleavage domain (CD) of a zinc finger nuclease (ZFN) having enhanced catalytic activity as compared to a reference ZFN, comprising:
a) expressing a mutated zinc finger nuclease (ZFN) comprising a DNA cleavage domain (CD) having one or more mutations, and a DNA binding zinc finger domain (ZFD) in a cell comprising a reporter construct,
wherein the reporter construct comprises in 5′ to 3′ order a promoter, a toxic gene, and a zinc finger nuclease cleavage site that is recognized by the ZFN, wherein the toxic gene is operatively linked to the promoter,
whereby the ZFN cleaves the reporter construct, thereby allowing the reporter construct comprising the toxic gene to be degraded; and
b) determining a survival rate for the cell, wherein survival rate is positively correlated with catalytic activity of the CD of the ZFN, and wherein a survival rate for a cell expressing the mutated ZFN that is higher than a survival rate of a cell expressing a reference ZFN is indicative of the CD of the mutated ZFN having enhanced catalytic activity.

2. The method of claim 1, wherein a reference ZFN comprises a wild type form of the CD having the one or more mutations.

3. The method of claim 1, wherein the mutated ZFN is encoded by an expression construct.

4. The method of claim 1, wherein the one or more mutations in the CD is introduced into a polynucleotide encoding the CD by error-prone PGR amplification of the polynucleotide.

5. The method of claim 1, further comprising transfecting the cell with an expression construct encoding the mutated ZFN.

6. The method of claim 1, wherein the cell is a bacterial cell.

7. The method of claim 6, wherein the bacterial cell is E. coli.

8. The method of claim 7, wherein the determining the survival rate of the cell step is performed by plating on selective medium the cell expressing the mutated ZFN and comparing to the number of colonies produced to the number of colonies produced by plating cells expressing the reference ZFN.

9. The method of claim 8, further comprising isolating the expression construct encoding the mutated ZFN; mutating the polynucleotide encoding the mutated ZFN to produce a second mutated ZFN and repeating the expressing and determining steps with the second mutated ZFN to identify altered catalytic activity in the second mutated ZFN, as compared to the mutated ZFN.

10. The method of claim 1, wherein the DNA cleavage domain is obtained from a FokI endonuclease.

11. The method of claim 1, wherein the toxic gene comprises the ccdB gene.

12. The method of claim 1, wherein the reporter construct comprises a BAD promoter.

13. A method of identifying a zinc finger nuclease (ZFN) having enhanced catalytic activity comprising:
a) subjecting a polynucleotide encoding a DNA cleavage domain (CD) to mutagenesis to produce mutated polynucleotides encoding CDs having one or more mutations;
b) fusing the mutated polynucleotides encoding the CDs having one or more mutations to a polynucleotide encoding a DNA binding zinc finger domain (ZFD), thereby creating a library of polynucleotides encoding mutated ZFNs;
c) expressing the library in cells that comprise a reporter construct,
wherein the reporter construct comprises in 5′ to 3′ order a promoter, a toxic gene, and a zinc finger nuclease cleavage site that is recognized by the ZFN, wherein the toxic gene is operatively linked to the promoter,
whereby the ZFN cleaves the reporter construct, thereby allowing the reporter construct comprising the toxic gene to be degraded; and
b) selecting cells expressing a mutated ZFN having a survival rate that is higher than a survival rate of a cell expressing a reference ZFN, wherein a higher survival rate is indicative of the mutated ZFN having enhanced catalytic activity.

14. The method of claim 13, wherein a reference ZFN comprises a wild type form of the CD having the one or more mutations.

15. The method of claim 13, wherein the mutagenesis is performed by error-prone PCR amplification of the polynucleotide encoding the mutated ZFN.

16. The method of claim 13, wherein the cell is a bacterial cell.

17. The method of claim 16, wherein the bacterial cell is E. coli.

18. The method of claim 13, wherein the determining the survival rate of the cell step is performed by plating on selective medium the cell expressing the mutated gene and comparing the number of colonies produced to the number of colonies produced by plating cells expressing the reference ZFN.

19. The method of claim 18, further comprising isolating the polynucleotide encoding the mutated ZFN; mutating the polynucleotide encoding the mutated CD to produce a second generation mutated CD and repeating the expressing and selecting steps with the second generation mutated ZFN to identify altered catalytic activity in the second generation mutated ZFN, as compared to the mutated ZFN.

20. The method of claim 19, further comprising repeating the isolating, mutating, expressing, and selecting steps one or more times to obtain successive generations of mutated ZFNs having altered catalytic activity as compared a prior generation mutated ZFN.

21. The method of claim 13, wherein the DNA cleavage domain is obtained from a FokI endonuclease.

22. The method of claim 13, wherein the toxic gene comprises the ccdB gene.

23. The isolated protein of claim 13, wherein the ZFD comprises three, or four, or more zinc finger proteins.

24. The method of claim 13, wherein the reporter construct comprises a BAD promoter.

25. The method of claim 13, further comprising mutating the polynucleotide encoding the DNA binding ZFD.

26. The method of claim 13, further comprising isolating the polynucleotide encoding the mutated ZFN; subjecting the polynucleotide encoding the mutated ZFN DNA shuffling to produce a second generation mutated ZFN and repeating the expressing and selecting steps with the second generation mutated ZFN to identify altered catalytic activity in the second generation mutated ZFN, as compared to the mutated ZFN.

27. An isolated zinc finger nuclease (ZFN) protein comprising a zinc finger DNA cleavage domain (CD) having altered catalytic activity obtained by the method of claim 1, and a DNA binding zinc finger domain (ZFD).

28. The isolated protein of claim 27, wherein the CD has enhanced catalytic activity as compared to the reference ZFN.

29. The isolated protein of claim 27, wherein the ZFD comprises three, or four, or more zinc finger proteins.

30. The isolated protein of claim 27, wherein the ZFD binds a specific sequence within a gene of interest.

31. An isolated zinc finger nuclease (ZFN) having altered catalytic activity obtained by the method of claim 13.

32. An isolated zinc finger nuclease comprising the amino acid sequence of SEQ ID NOs:1 or 2.

33. An isolated zinc finger nuclease comprising a DNA binding zinc finger domain (ZFD) and a zinc finger DNA cleavage domain (CD) selected from the group consisting of SEQ ID NO:3, SEQ ID NO:4, SEQ ID NO:5, and SEQ ID NO:6.

34. A method of introducing a break into a nucleic acid molecule at a site of interest comprising:
contacting a nucleic acid molecule with a ZFN of claim 27, wherein the ZFN comprises a DNA binding zinc finger domain (ZFD) that binds a target site in proximity to the site of interest so that upon binding of the ZFN to the target site, the ZFN cleaves the nucleic acid at the site of interest, thereby introducing a break into the nucleic acid molecule.

35. The method of claim 34. wherein the nucleic acid molecule is genomic DNA and the break is a double stranded break in the nucleic acid molecule.

36. The method of claim 35, wherein the double stranded break results in inactivation of a gene of interest.

37. A method of treating a subject having a cell proliferative disorder, comprising:
inactivating or mutating a gene according to the method of claim 34 in one or more cells of the subject, wherein over-expression of the gene is associated the cell proliferative disorder, thereby treating the cell proliferative disorder.

38. The method of claim 37, wherein the cell proliferative disorder is a cancer.

39. A method of producing a cell in which a gene of interest has been mutated comprising:
mutating the gene of interest in a cell or population of cells by introducing, into the cells, a ZFN of any claim 27, wherein the ZFN comprises a DNA binding zinc finger domain (ZFD) that binds a target site within the gene of interest, such that the ZFN is expressed in the cell, whereby the ZFN binds to the target site and cleaves the gene of interest; and
culturing the cells whereby progeny cells in which the gene of interest is mutated are produced.

40. The method of claim 39, wherein the cell is transfected with a nucleic acid molecule encoding the ZFN.

41. A method of mutating or knocking out a gene of interest in a cell or population of cells comprising:
mutating the gene of interest in a target cell by contacting the cell with a ZFN protein of claim 27 or containing a CD of native or engineered sequence, wherein the ZFD binds a target site within the cell genome, with the proviso that the ZFN is not fused or conjugated to a protein transduction domain, such that the ZFN hinds to the target site and cleaves the gene of interest; and
culturing the cell, whereby progeny cells in which the gene of interest is mutated or knocked out are produced.

42. The method of claim 41, wherein mutating the gene of interest results in activation or restoration of expression of the gene of interest.

43. The method of claim 41, comprising delivering to the cell, either prior to, simultaneously with or following mutating the gene of interest, a corrective nucleic acid or vector containing the nucleic acid, thereby providing a substitute for the knocked out or mutated gene of interest.

44. A method of mutating a gene of interest in a cell or population of cells comprising:
mutating the gene of interest in a target cell by contacting the cell with a ZFN protein of claim 27 or containing a CD of engineered sequence, wherein the ZFD binds a target site within the cell genome, and wherein the ZFN is fused or conjugated to a protein transduction domain, such that the ZFN binds to the target site and cleaves the gene of interest; and
culturing the cell, whereby progeny cells in which the gene of interest is mutated or knocked out are produced.

45. The method of claim 44, wherein mutating the gene of interest results in activation or restoration of expression of the gene of interest.

46. The method of claim 44, comprising delivering to the cell, either prior to, simultaneously with or following mutating the gene of interest, a corrective nucleic acid or vector containing the nucleic acid, thereby providing a substitute for the knocked out or mutated gene of interest.


SEQUENCE LISTING (29 sequences)

SEQ ID NO: 1 - protein, 284 aa, Homo sapiens
MEKPYACPECGKSFSRSDDLVRHQRTHTGEKPYKCPECGKSFSRSDVLVRHQRTHTGEKPYKCPECGKSFSQSGDLRRHQRTHTGKKTQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 2 - protein, 395 aa, Homo sapiens
MPKKKRKVEDPRFMYPYDVPDYAGLEPGEKPYACPECGKSFSRKDSLVRHQRTHTGEKPYKCPECGKSFSQSGDLRRHQRTHTGEKPYKCPECGKSFSDCRDLARHQRTHTGEKPYACPECGKSFSQSSHLVRHQRTHTGEKPYKCPECGKSFSDCRDLARHQRTHTGEKPYKCPECGKSFSRSDKLVRHQRTHTGKKTQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 3 - protein, 196 aa, Homo sapiens
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 4 - protein, 196 aa, Artificial Sequence
  Synthetic construct
QLVKSELEEKKSELRHKLKYVPHEYIELIEIARNPTQDRILEMKVMEFLMKVYGYRGEHLGGSRKPDGAIYTVGSPIDYGVIVDTKAYSGGYNLPIGHADEMQRYVEENQTRNKHINPNEWWKVYPSSVTEFKFLFVSGYFKGDYKAQLTRLNHITNCNGAVLSVEELLIGGEMIQAGTLTLEEVRRKFNNGEINF

SEQ ID NO: 5 - protein, 196 aa, Artificial Sequence
  Synthetic construct
QLVKSELEEKKSELRHKLKYVPHEYIELIEIARNPTQDRILEMKVMEFLMKVYGYRGEHLGGSRKPDGAIYTVGSPIDYGVIVDTKAYSGGYNLPIGHAREMQRYVEENQTRNKHINPNEWWKVYPSSVTEFKFLFVSGYFKGDYKAQLTRLNHITNCNGAVLSVEELLIGGEMIQAGTLTLEEVRRKFNNGEINF

SEQ ID NO: 6 - protein, 195 aa, Artificial Sequence
  Synthetic construct
QLVKSELEEKKSELRHKLKYVPHEYIELIEIARNPTQDRILEMKVMEFLMKVYGYRGEHLGGSRKPDGAIYTVGSPIDYGVIVDTKAYSGGYNLPIGHADAMQSYVEENQTRNKHINPNEWWKVYPSSVTEFKFLFVSGYFKGDYKAQLTRLNHITNCNGAVLSVEELLIGGEMIQAGTLTLEEVRRKFNNGEIN

SEQ ID NO: 7 - protein, 334 aa, Artificial Sequence
  Synthetic construct
MHHHHHHASYPYDVPDYAGLEPGEKPYKCPECGKSFSDCRDLARHQRTHTGEKPYACPECGKSFSQSSHLVRHQRTHTGEKPYKCPECGKSFSDCRDLARHQRTHTGEKPYKCPECGKSFSRSDKLVRHQRTHTGKKTQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 8 - DNA, 77 bp, Artificial Sequence
  Synthetic construct
tcgccggcgattaccctgttatccctacactgcggctccggccccacgcgtggggccggagccgcagtgcaccggtg

SEQ ID NO: 9 - DNA, 77 bp, Artificial Sequence
  Synthetic construct
agcggccgctaatgggacaatagggatgtgacgccgaggccggggtgcgcaccccggcctcggcgtcacgtggccac

SEQ ID NO: 10 - protein, 4 aa, Artificial Sequence
  Synthetic construct
CCHH

SEQ ID NO: 11 - protein, 25 aa, Artificial Sequence
  Synthetic construct
CXXXXCXXXXXXXXXXXXHXXXXXH

SEQ ID NO: 12 - protein, 5 aa, Artificial Sequence
  Synthetic construct
TGEKP

SEQ ID NO: 13 - DNA, 56 bp, Artificial Sequence
  Primer
cactctagacgccactgcacgcgtgcagtggcgctagggataacagggtaatatag

SEQ ID NO: 14 - DNA, 35 bp, Artificial Sequence
  Primer
caccacgcatgcctatattaccctgttatccctag

SEQ ID NO: 15 - DNA, 65 bp, Artificial Sequence
  Primer
cactctagacgccactgcacgcgtggggccggagccgcagtgctagggataacagggtaatatag

SEQ ID NO: 16 - DNA, 35 bp, Artificial Sequence; identical to SEQ ID NO: 14
  Primer

SEQ ID NO: 17 - DNA, 12 bp, Artificial Sequence
  Synthetic construct
ggggccggagcc

SEQ ID NO: 18 - DNA, 15 bp, Artificial Sequence
  Synthetic construct
ggggccggagccgca

SEQ ID NO: 19 - DNA, 18 bp, Artificial Sequence
  Synthetic construct
ggggccggagccgcagtg

SEQ ID NO: 20 - DNA, 24 bp, Artificial Sequence
  Synthetic construct
tccggccccacgcgtggggccgga

SEQ ID NO: 21 - DNA, 24 bp, Artificial Sequence
  Synthetic construct
cgccactgcacgcgtgcagtggcg

SEQ ID NO: 22 - DNA, 33 bp, Artificial Sequence
  Synthetic construct
cgccactgcacgcgtggggccggagccgcagtg

SEQ ID NO: 23 - DNA, 30 bp, Artificial Sequence
  Synthetic construct
ggctccggccccacgcgtggggccggagcc

SEQ ID NO: 24 - DNA, 36 bp, Artificial Sequence
  Synthetic construct
tgcggctccggccccacgcgtggggccggagccgca

SEQ ID NO: 25 - DNA, 42 bp, Artificial Sequence
  Synthetic construct
cactgcggctccggccccacgcgtggggccggagccgcagtg

SEQ ID NO: 26 - protein, 4 aa, Artificial Sequence
  Synthetic construct
GEKP

SEQ ID NO: 27 - protein, 4 aa, Artificial Sequence
  Synthetic construct
GEEP

SEQ ID NO: 28 - protein, 4 aa, Artificial Sequence
  Synthetic construct
GKKT

SEQ ID NO: 29 - protein, 5 aa, Artificial Sequence
  Synthetic construct
DAMQS